(19) United States
(12) Patent Application Publication
(10) Pub. No.: US 2023/0183181 A1
(43) Pub. Date: Jun. 15, 2023

(54) PROCESS OF PREPARING BUTYL-(5S)-5-({2-[4-(BUTOXYCARBONYL)PHENYL]ETHYL}[2-(2-{[3-CHLORO-4'-(TRIFLUOROMETHYL)[BIPHENYL]-4-YL]METHOXY}PHENYL)ETHYL]AMINO)-5,6,7,8-TETRAHYDROQUINOLINE-2-CARBOXYLATE

(71) Applicants: BAYER AKTIENGESELLSCHAFT, Leverkusen (DE); BAYER PHARMA AKTIENGESELLSCHAFT, Berlin (DE)

(72) Inventors: Peter FEY, Wuppertal (DE); Marc NOWAKOWSKI, Wuppertal (DE); Julian EGGER, Remscheid (DE); Nadine BREMEYER, Düsseldorf (DE)

(21) Appl. No.: 17/999,179

(22) PCT Filed: May 14, 2021
(86) PCT No.: PCT/EP2021/062837

§ 371 Date: Nov. 17, 2022

(30) Foreign Application Priority Data
May 20, 2020 (EP) 20175721.8

(51) Int. Cl. C07D 221/04

(52) U.S. Cl. CPC C07D 221/04

(57) ABSTRACT

The present invention relates to a novel and improved process for preparing butyl (5S)-5-({2-[4-(butoxycarbonyl)phenyl]ethyl}[2-(2-{[3-chloro-4′-(trifluoromethyl)[biphenyl]-4-yl]methoxy}phenyl)ethyl]amino)-5,6,7,8-tetrahydroquinoline-2-carboxylate of the formula (XII)

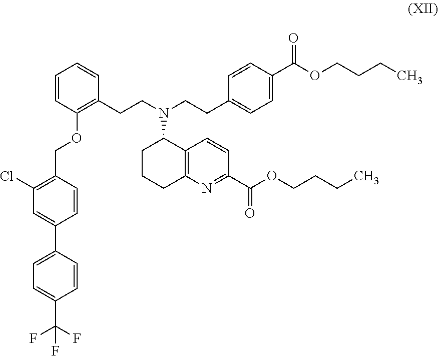 [SMILES: CCCCOC(=O)c1ccc(CCN(CCc2ccccc2OCc2ccc(-c3ccc(C(F)(F)F)cc3)cc2Cl)[C@H]2CCCc3nc(C(=O)OCCCC)ccc32)cc1]

to novel precursors for preparation thereof, and to use for preparation of (5S)-5-{[2-(4-carboxyphenyl)ethyl][2-(2-{[3-chloro-4′-(trifluoromethyl)[biphenyl]-4-yl]methoxy}phenyl)ethyl]amino}-5,6,7,8-tetrahydroquinoline-2-carboxylic acid.

The present invention relates to a novel and improved process for preparing butyl (5S)-5-({2-[4-(butoxycarbonyl)phenyl]ethyl}[2-(2-{[3-chloro-4′-(trifluoromethyl)[biphenyl]-4-yl]methoxy}phenyl)ethyl]amino)-5,6,7,8-tetrahydroquinoline-2-carboxylate of the formula (XII)

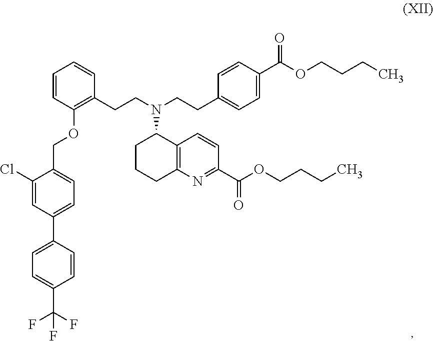 [SMILES: CCCCOC(=O)c1ccc(CCN(CCc2ccccc2OCc2ccc(-c3ccc(C(F)(F)F)cc3)cc2Cl)[C@H]2CCCc3nc(C(=O)OCCCC)ccc32)cc1]

to novel precursors for preparation thereof, and to use for preparation of (5S)-5-{[2-(4-carboxyphenyl)ethyl][2-(2-{[3-chloro-4′-(trifluoromethyl)[biphenyl]-4-yl]methoxy}phenyl)ethyl]amino}-5,6,7,8-tetrahydroquinoline-2-carboxylic acid.

The compound of the formula (XII) is a precursor of (5S)-5-{[2-(4-carboxyphenyl)ethyl][2-(2-{[3-chloro-4′-(trifluoromethyl)I[biphenyl]-4-yl]methoxy}phenyl)ethyl]amino}-5,6,7,8-tetrahydroquinoline-2-carboxylic acid of the formula (I)

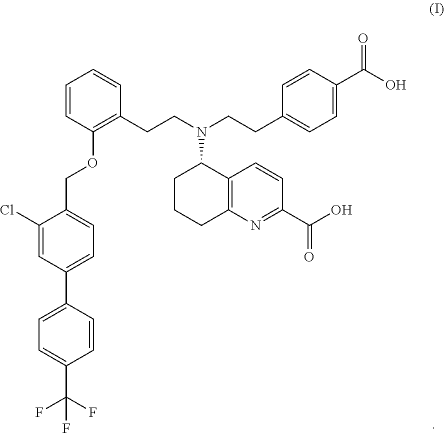 [SMILES: O=C(O)c1ccc(CCN(CCc2ccccc2OCc2ccc(-c3ccc(C(F)(F)F)cc3)cc2Cl)[C@H]2CCCc3nc(C(=O)O)ccc32)cc1]

The compound of the formula (XII) can be converted to the compound of the formula (I) by ester hydrolysis.

The compound of the formula (I) acts as activator of soluble guanylate cyclase and can be used as an agent for prophylaxis and/or treatment of pulmonary, cardiopulmonary and cardiovascular disorders, for example for treatment of pulmonary arterial hypertension (PAH), pulmonary hypertension (PH), pulmonary hypertension associated with chronic obstructive lung disease (PH-COPD), pulmonary hypertension associated with idiopathic interstitial pneumonia (PH-IIP) or chronic thromboembolic pulmonary hypertension (CTEPH).

The compound of the formula (I) and a preparation process are described in WO 2014/012934. A disadvantage of the synthesis described in WO 2014/012934 is the fact that this synthesis is unsuitable for an industrial scale process since, among other reasons, seven chromatographic purification steps and one chiral chromatography stage are needed for separation of enantiomers of one racemate. These are generally technically highly complex and costly and require a large solvent consumption, and should therefore be avoided if possible. In addition, separation into enantiomers takes place at an advanced stage of the synthesis by chromatography on chiral phase. This gives rise to a high proportion of product that cannot be used for further synthesis.

Some stages of the synthesis described in WO 2014/012934 also feature a long reaction time over several days and a low yield, which is a considerable disadvantage for the efficiency of a synthesis on the industrial scale. For example, the reaction time is four days for preparation of Example 6A, and three days for preparation of Example 92A. In addition, the use of the excess of methyl 4-(2-iodoethyl)benzoate in the preparation of Example 92A can lead to polymerization. This forms polystyrene, which has to be removed in a complex manner.

Some stages are not implementable on an industrial scale owing to safety- and process-related difficulties. Some reaction stages proceed in very high dilution and with use of very large amounts of reagent, which means that little product can be produced relative to the volume of a batch. In addition, the synthesis disclosed in WO 2014/012934 consists of 17 stages, and for that reason alone is very costly and time-consuming.

There was therefore a need for a synthesis practicable on an industrial scale that affords the compounds of the formula (I) reproducibly in a high overall yield, with low production costs and high purity, and meets all regulatory requirements.

It is a feature of the process according to the invention that steps for purification of the intermediates take place by salt formation, and it is therefore possible to dispense with chromatographic purification steps. Enantioselective synthesis means that no chiral chromatography stage is necessary for separation of enantiomers of a racemate. The number of synthesis stages in the process according to the invention has been reduced compared to the synthesis disclosed in WO 2014/012934.

The process according to the invention is therefore suitable for preparing the compound of the formula (I) reproducibly and in a high overall yield and purity, in a synthesis practicable on an industrial scale.

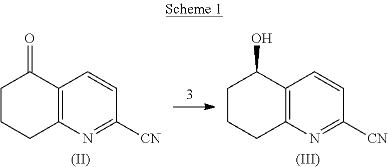

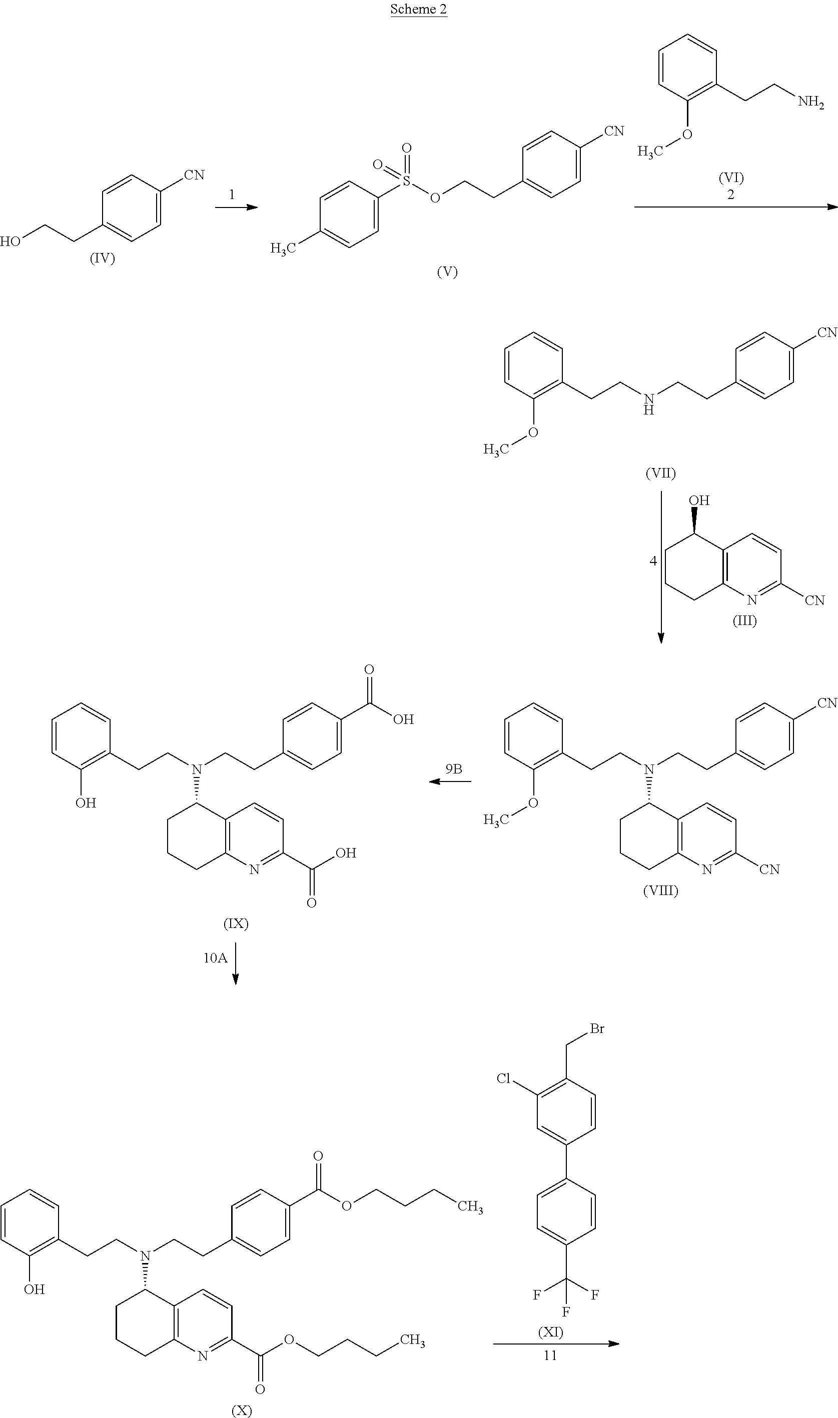

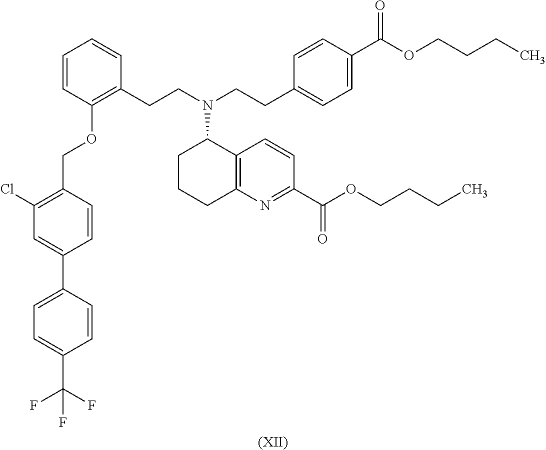

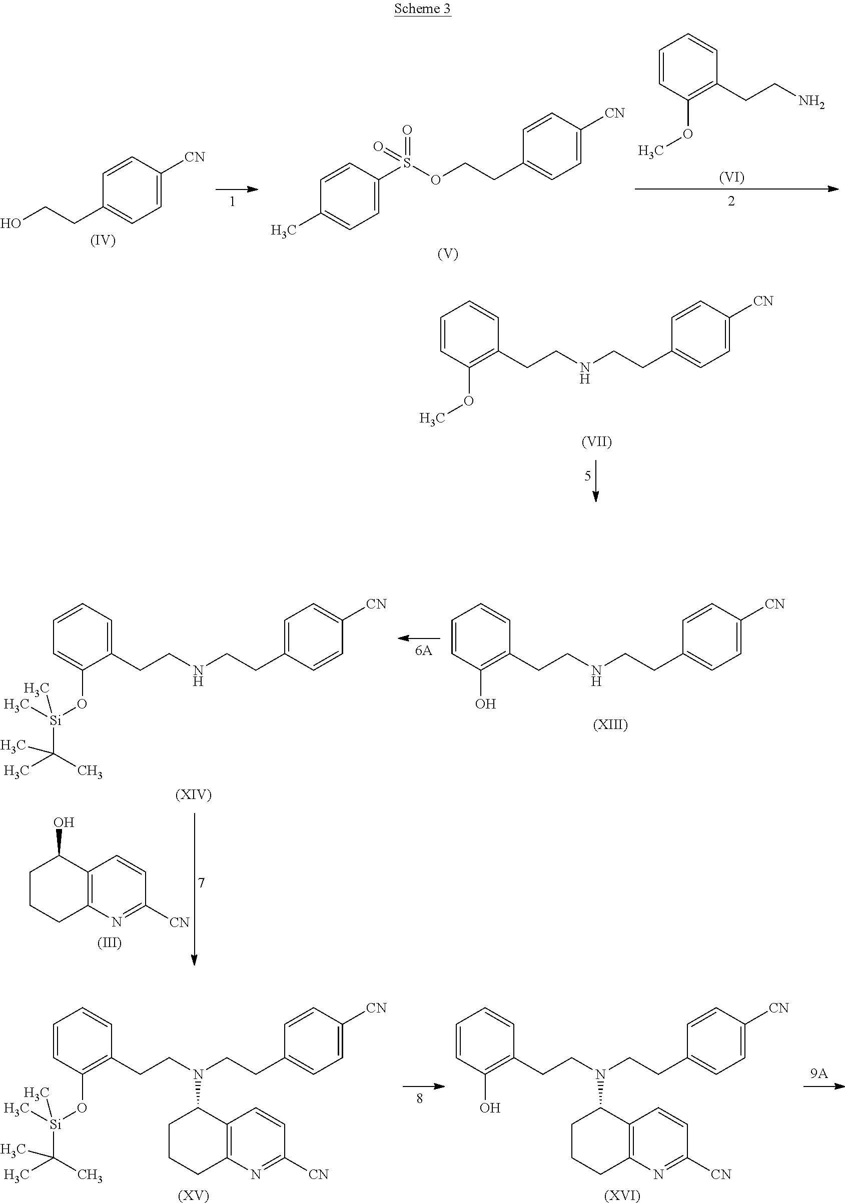

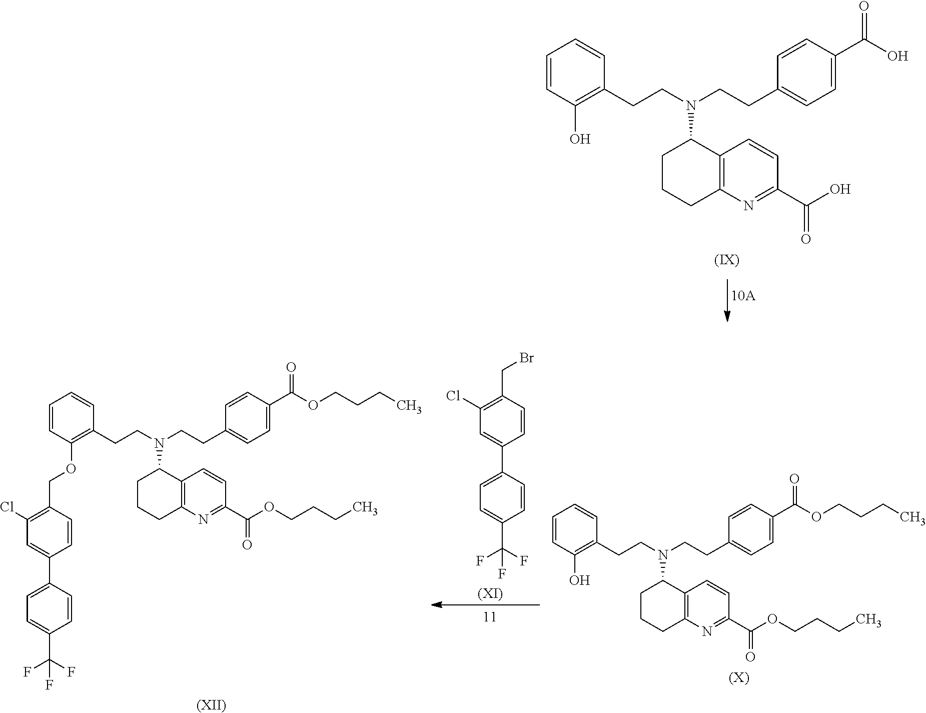

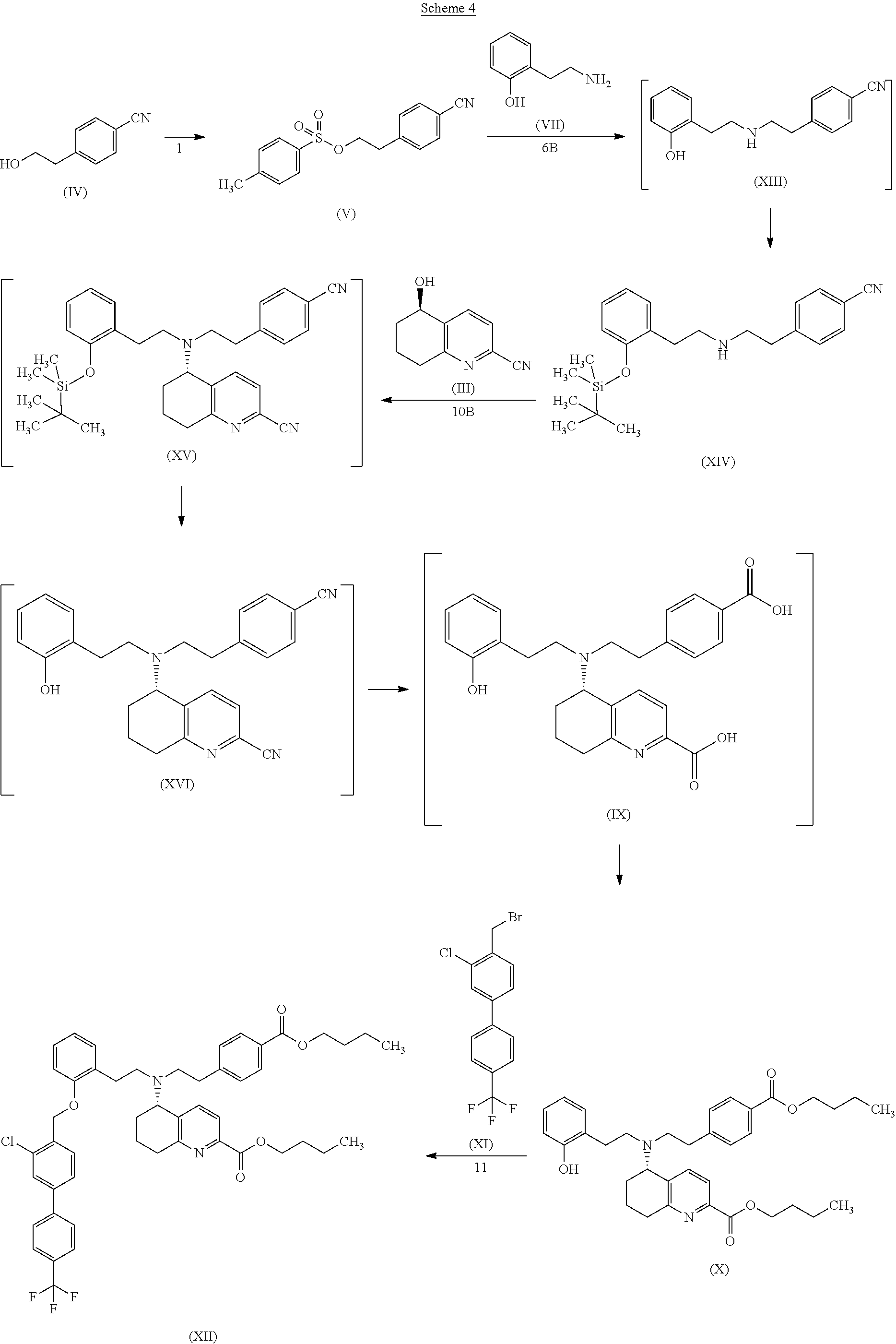

Scheme 1 shows the preparation of the compound of the formula (III) which is required for preparation of the compound of the formula (XII).

Scheme 2 shows an overview of the synthesis steps for preparation of the compound of the formula (XII) via the intermediate of the compound of the formula (VIII).

Scheme 3 shows an overview of the synthesis steps for preparation of the compound of the formula (XII) via the intermediate of the compound of the formula (XV).

Scheme 4 shows an overview of the synthesis steps for preparation of the compound of the formula (XII), wherein the reaction regime is analogous to that shown in Scheme 3, except that various intermediate stages are not isolated.

### DESCRIPTION OF THE INDIVIDUAL SYNTHESIS STAGES

### Example 1

Process Step 1

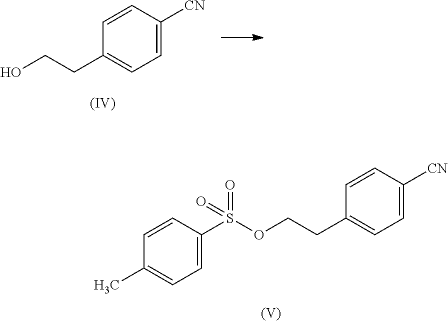

Process step 1 (Schemes 2 and 3) describes the preparation of 2-(4-cyanophenyl)ethyl 4-methylbenzenesulfonate of the formula (V) from 4-(2-hydroxyethyl)benzonitrile of the formula (IV). The compound of the formula (IV), potassium hydroxide and 4-toluenesulfonyl chloride (TsCl) are added here to an inert solvent, for example suitable ethers such as 2-methyltetrahydrofuran (2-MTHF), tetrahydrofuran (THF) or dioxane, preferably THF, and stirred. The temperature is kept between −10° C. and 0° C. until all compounds have been added, in order to avoid elimination reactions that lead to cyanostyrenes and polymerization products thereof. This is followed by stirring at a temperature of 0° C. to 30° C., preferably 22° C., until conversion is complete.

The compound of the formula (V) can be isolated, for example, by aqueous workup and subsequent crystallization. Suitable methods of aqueous workup are extractions that are known to the person skilled in the art and are capable of separating off by-products and excess potassium hydroxide. Aqueous workup can be effected, for example, with dichloromethane (DCM) and water in the presence of ammonium chloride. Crystallization may take place, for example, in cyclohexane. This involves changing the solvent to cyclohexane, concentrating under reduced pressure at a temperature of 30° C. to 50° C., preferably 41° C., cooling to a temperature of 20° C. to 30° C., preferably 22° C., isolating the solids and drying in a drying cabinet at a temperature of 30° C. to 50° C., preferably 40° C.

The present invention provides a process for preparing the compound of the formula (V)

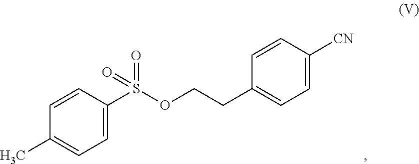 [SMILES: Cc1ccc(S(=O)(=O)OCCc2ccc(C#N)cc2)cc1]

characterized in that the compound of the formula (IV)

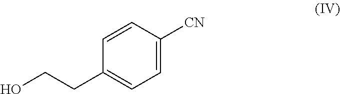 [SMILES: N#Cc1ccc(CCO)cc1]

is reacted with potassium hydroxide and 4-toluenesulfonyl chloride in an inert solvent.

The present invention further provides a process for preparing the compound of the formula (V) as described above, wherein the inert solvent is an ether selected from a list comprising 2-methyltetrahydrofuran, tetrahydrofuran or dioxane, preferably tetrahydrofuran.

The present invention further provides a process for preparing the compound of the formula (V) as described above, wherein the temperature on addition of the compound of the formula (IV), potassium hydroxide and 4-toluenesulfonyl chloride is kept between −10° C. and 0° C.

The present invention further provides a process for preparing the compound of the formula (V) as described above, wherein conversion is effected at a temperature of 0° C. to 30° C., preferably 22° C.

### Example 2

Process Step 2

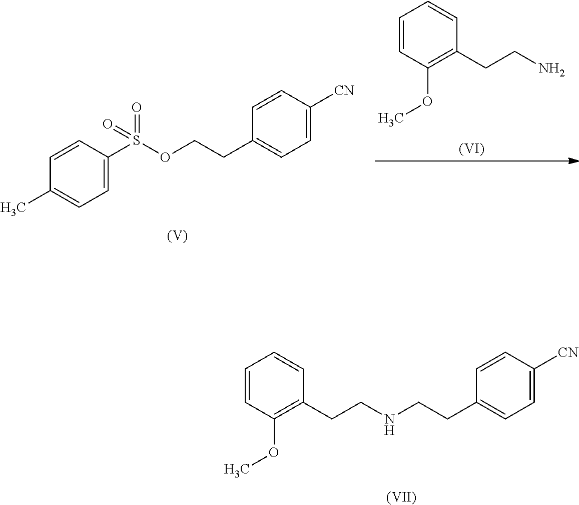 [SMILES: Cc1ccc(S(=O)(=O)OCCc2ccc(C#N)cc2)cc1 ; COc1ccccc1CCN ; "(VI)" ; "(V)" ; COc1ccccc1CCNCCc1ccc(C#N)cc1 ; "(VII)"]

For preparation of 4-(2-{[2-(2-methoxyphenyl)ethyl]amino}ethyl)benzonitrile of the formula (VII) by process step 2 (Schemes 2 and 3), 2-(4-cyanophenyl)ethyl 4-methylbenzenesulfonate of the formula (V) is suspended in a suitable ether, preferably THF, and 2-methoxyphenethylamine of the formula (VI) and a tertiary amine base, for example and with preference triethylamine, are added and heated under reflux, preferably for 2 h. Subsequently, the solvent is changed to water and a mineral acid, preferably hydrochloric acid, more preferably 25% hydrochloric acid, is added at a temperature of 0 to 30° C. The solids in the reaction mixture are isolated.

The compound of the formula (VII) is preferably isolated as an oil after an aqueous workup. Suitable methods of aqueous workup are extractions that are known to the person skilled in the art and are capable of separating off by-products, for example excess toluenesulfonic acid. By way of example and with preference, the isolated solids are admixed with water and stirred, and then the solids are filtered off. This operation can be performed repeatedly. The solids are preferably admixed with ethyl acetate at a temperature of 30 to 60° C., more preferably 50° C., and stirred, and the solids are preferably isolated at a temperature of 10 to 30° C., more preferably 20° C. This operation can be performed repeatedly, before the solids are dried under reduced atmospheric pressure, preferably at a temperature of 40° C. In a further step, the solids are admixed with a mixture of ethyl acetate and hydrochloric acid, preferably 15% hydrochloric acid, in order to obtain the hydrochloride of the compound of the formula (VII), which is dried under reduced atmospheric pressure, preferably at a temperature of 40° C. In order to obtain the free base of the compound of the formula (VII), the solids obtained are dissolved in DCM and water, preferably in equal proportions by volume, and adjusted to a pH between 13 and 14 with an alkali, preferably sodium hydroxide solution, more preferably 45% sodium hydroxide solution. The organic phase is isolated, washed with water and concentrated under reduced atmospheric pressure, preferably at a temperature of 40° C., to give an oil.

Alkylation reactions of primary amines generally afford mixtures of the possible polyalkylation products. It is an advantage of this process that the desired monoalkylation product VII is obtained in good purity and yield under the optimized reaction and workup conditions. The polyalkylation products that are formed here too are successfully removed by the optimized purification.

The present invention further provides the compound of the formula (VII)

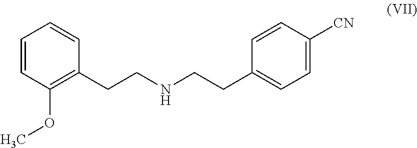 [SMILES: COc1ccccc1CCNCCc1ccc(C#N)cc1]

and the salts, solvates and solvates of the salts thereof.

The present invention further provides the oxalate salt of the compound of the formula (VII).

The present invention further provides a process for preparing the compound of the formula (VII), characterized in that the compound of the formula (V)

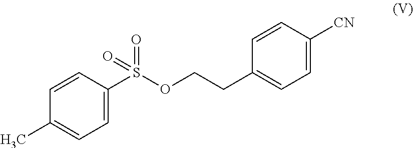 [SMILES: Cc1ccc(S(=O)(=O)OCCc2ccc(C#N)cc2)cc1]

in a first step, suspended in a suitable ether in the presence of a tertiary amine base, is reacted with the compound of the formula (VI)

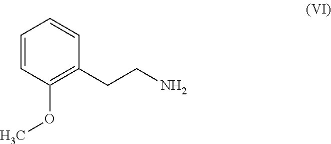

and in a second step the solvent is changed to water and a mineral acid is added.

The present invention further provides a process for preparing the compound of the formula (VII) as described above, wherein the suitable ether is tetrahydrofuran.

The present invention further provides a process for preparing the compound of the formula (VII) as described above, wherein the tertiary amine base is triethylamine.

The present invention further provides a process for preparing the compound of the formula (VII) as described above, wherein the reaction takes place at reflux temperature in the first step.

The present invention further provides a process for preparing the compound of the formula (VII) as described above, wherein the second step takes place at a temperature of 0° C. to 30° C.

The present invention further provides a process for preparing the compound of the formula (VII) as described above, wherein the mineral acid is hydrochloric acid, preferably 25% hydrochloric acid.

### Example 3

Process Step 3

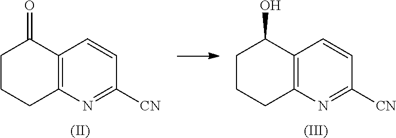

The prior art describes the preparation of the compound of the formula (I) by reductive amination of 5-oxo-5,6,7,8-tetrahydroquinoline-2-carbonitrile (II) with the amine of the formula XVII and subsequent alkylation reaction, resulting in a racemic end product. Subsequently, it is necessary to separate the enantiomers in a chiral chromatography stage. This is technically very complex and costly, and entails a high consumption of solvents. In the present invention, an effective process has surprisingly been found for preparation of (5R)-5-hydroxy-5,6,7,8-tetrahydroquinoline-2-carbonitrile (compound of the formula (III)). With the aid of the compound of the formula (III), it is possible to obtain an enantiomerically pure end product, which avoids the disadvantageous chiral chromatography stage.

For preparation of (5R)-5-hydroxy-5,6,7,8-tetrahydroquinoline-2-carbonitrile of the formula (III) in process step 3 (Scheme 1), 5-oxo-5,6,7,8-tetrahydroquinoline-2-carbonitrile of the formula (II) (preparation disclosed in WO 2014/12934 as Example 4A) is initially charged in a suitable solvent.

Suitable solvents are esters known as solvents to the person skilled in the art, for example ethyl acetate, and ethers, for example diethyl ether, dioxane, tetrahydrofuran; preference is given to using ethyl acetate. At a temperature of preferably 0 to 40° C., more preferably 20° C., a tertiary amine base, for example with preference triethylamine and ruthenium-p-cymene-R,R-TsDPEN (CAS number: 192139-92-7), is added, preferably in catalytic amounts. At a temperature of preferably −5 to 10° C., more preferably 0 to 5° C., formic acid is added, and gases formed are removed. Stirring is continued at a temperature of preferably 20 to 50° C., more preferably 40° C., until conversion is complete.

The compound of the formula (III) is preferably isolated after workup and subsequent crystallization. For workup, the reaction mixture is admixed and stirred with preferably equal volumes of a mixture of ethyl acetate and a mineral acid, preferably hydrochloric acid, more preferably 1 N hydrochloric acid, and the upper phase is isolated. A C₆-C₈-alkane, preferably heptane, more preferably n-heptane, is added to the upper phase, and the mixture is concentrated under reduced atmospheric pressure, preferably at a temperature of 20 to 50° C., more preferably 40° C. This step can be performed repeatedly. At a preferred temperature of 20° C., the compound of the formula (III) is isolated from the mixture in solid form and dried, preferably at reduced pressure at a temperature of 40° C.

The present invention further provides (5R)-5-hydroxy-5,6,7,8-tetrahydroquinoline-2-carbonitrile of the formula (III)

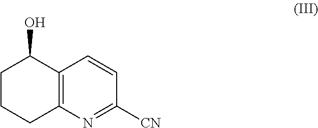 [SMILES: N#Cc1ccc2c(n1)CCC[C@H]2O]

and the salts, solvates and solvates of the salts thereof.

The present invention further provides a process for preparing the compound of the formula (III), characterized in that the compound of the formula (II)

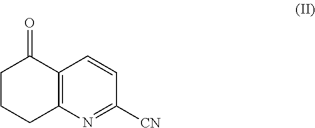 [SMILES: N#Cc1ccc2c(n1)CCCC2=O]

is reacted with a tertiary amine base, ruthenium-p-cymene-R,R-TsDPEN and formic acid to give the compound of the formula (III).

The present invention further provides a process for preparing the compound of the formula (III) as described above, wherein the amine base is triethylamine, and ruthenium-p-cymene-R,R-TsDPEN is used in catalytic amounts.

The present invention further provides a process for preparing the compound of the formula (III) as described above, wherein the compound of the formula (II), before the reaction, is dissolved in a solvent selected from a list comprising ethyl acetate, diethyl ether, dioxane and tetrahydrofuran, preferably ethyl acetate.

The present invention further provides a process for preparing the compound of the formula (III) as described above, wherein the compound of the formula (II) is admixed in a first step with the amine base and ruthenium-p-cymene-R,R-TsDPEN, and in a second step formic acid is added.

The present invention further provides a process for preparing the compound of the formula (III) as described above, wherein the compound of the formula (II) is admixed in a first step with the amine base and ruthenium-p-cymene-R,R-TsDPEN at a temperature of 0° C. to 40° C., preferably 20° C., and in a second step formic acid is added at a temperature of −5° C. to 10° C., preferably 0° C. to 5° C.

The present invention further provides a process for preparing the compound of the formula (III) as described above, wherein, after addition of the formic acid, stirring is continued at a temperature of 20° C. to 50° C., preferably 40° C., until conversion is complete.

### Example 4

Process Step 4

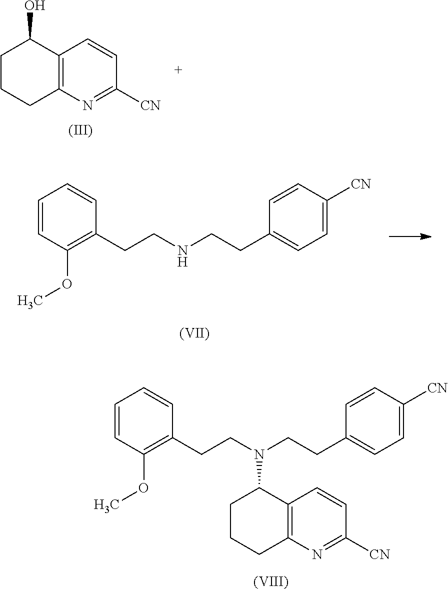

Process step 4 (Scheme 2) describes the preparation of (5S)-5-{[2-(4-cyanophenyl)ethyl][2-(2-methoxyphenyl)ethyl]amino}-5,6,7,8-tetrahydroquinoline-2-carbonitrile of the formula (VIII). For this purpose, preferably with exclusion of water, more preferably under a protective gas atmosphere, for example under sparging with argon, a solution of (5R)-5-hydroxy-5,6,7,8-tetrahydroquinoline-2-carbonitrile (III) is dissolved in a suitable solvent. Suitable solvents are those that are liquid at the reaction temperatures, for example THF or DCM; preference is given to using DCM. A suitable base is added to the solution. Suitable bases are sterically hindered secondary amines or 2,6-disubstituted pyridines, for example 2,6-lutidine or 2,6-di-tert-butylpyridine. Suitable sterically hindered secondary amines are, for example, diisopropylamine, 2,5-dimethylpiperidine or 2,2,5,5-tetramethylpiperidine. It is surprisingly possible with these compounds to achieve better yields compared to sterically unhindered or tertiary amines. It was especially unsurprising that the most advantageous yields are obtained by the use of diisopropylamine, preferably in a molar excess based on the compound of the formula (III). Diisopropylamine, being a secondary amine, is an unusual base for this type of reaction. The reaction mixture is cooled to a temperature between −90° C. and −50° C., preferably −78° C. and −65° C. While maintaining this temperature range, 4-(2-{[2-(2-methoxyphenyl)ethyl]amino}ethyl)benzonitrile of the formula (VII) is added, preferably in a molar ratio of 1:1 based on the compound of the formula (III), and stirred.

The compound of the formula (VIII) is preferably isolated after aqueous workup and subsequent crystallization. Suitable methods of aqueous workup are extractions that are known to the person skilled in the art and are capable of separating off by-products. For example and with preference, the reaction mixture, after complete conversion, may be admixed with a suitable acid, preferably oxalic acid or phosphoric acid, more preferably oxalic acid, and adjusted to a temperature of −10 to 15° C., preferably 0 to 5° C. Kieselguhr is added to the mixture, which is stirred. The solids are filtered off and discarded, and the liquid organic phase is washed with water and adjusted to a pH of 7.5 to 9, preferably 8, with a base, preferably ammonia solution, more preferably 27% ammonia solution. The organic phase is isolated and preferably concentrated under reduced atmospheric pressure to give an oil.

The compound of the formula (VIII) is crystallized by dissolving the oil in ethanol. At a temperature of 50° C. or less, preferably 40° C. or less, more preferably 40° C., the compound of the formula (VIII) crystallizes out, preferably after seeding. The solids are isolated and dried by methods known to those skilled in the art, preferably under reduced atmospheric pressure, at a temperature of 25° C. and in a stream of nitrogen.

The present invention further provides the compound of the formula (VIII)

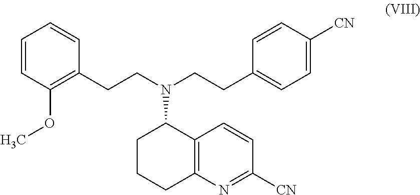 [SMILES: COc1ccccc1CCN(CCc1ccc(C#N)cc1)[C@H]1CCCc2nc(C#N)ccc21]

and the salts, solvates and solvates of the salts thereof.

The present invention further provides the compound of the formula (VIII-1)

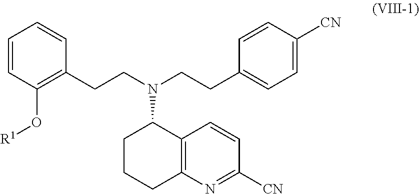

where

R¹ is C₁-C₄-alkyl

and the salts, solvates and solvates of the salts thereof.

The present invention further provides a process for preparing the compound of the formula (VIII-1)

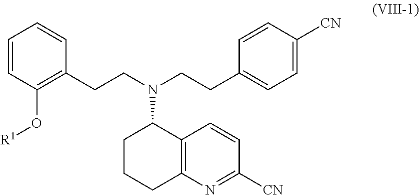

where

R¹ is C₁-C₄-alkyl,

characterized in that, at a temperature of −90° C. to −50° C. in a first step, it is added to the compound of the formula (III)

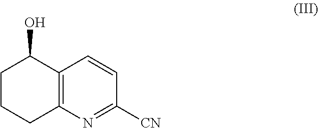 [SMILES: N#Cc1ccc2c(n1)CCC[C@H]2O]

in the presence of a base selected from a list comprising sterically hindered secondary amines and 2,6-disubstituted pyridines and trifluoromethanesulfonic anhydride, and in a second step is reacted with the compound of the formula (VII-1)

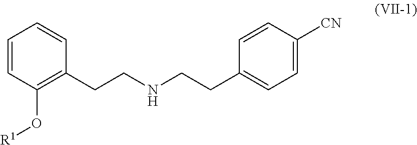

where

R¹ is C₁-C₄-alkyl.

In the context of the present invention, “C₁-C₄-alkyl” refers to a straight-chain or branched monovalent alkyl radical having 1 to 4 carbon atoms. Preferred examples include: methyl, ethyl, n-propyl, isopropyl, n-butyl, isobutyl, sec-butyl and tert-butyl.

The present invention further provides a process for preparing the compound of the formula (VIII-1) as described above, wherein R¹ is methyl.

The present invention further provides a process for preparing the compound of the formula (VIII-1) as described above, wherein the reaction takes place at a temperature of −78° C. to −65° C.

The present invention further provides a process for preparing the compound of the formula (VIII-1) as described above, wherein the sterically hindered secondary amine is selected from a list comprising diisopropylamine, 2,5-dimethylpiperidine and 2,2,5,5-tetramethylpiperidine.

The present invention further provides a process for preparing the compound of the formula (VIII-1), wherein the base is diisopropylamine.

The present invention further provides a process for preparing the compound of the formula (VIII-1), wherein the temperature is −78° C. to −65° C., preferably −76° C.

The present invention further provides a process for preparing the compound of the formula (VIII-1), wherein the compound of the formula (III) has been dissolved in tetrahydrofuran or dichloromethane, preferably dichloromethane.

The present invention further provides a process for preparing the compound of the formula (VIII-1) as described above, wherein the base is in a molar excess, preferably in a ratio of 3:1 based on the compound of the formula (III).

The present invention further provides a process for preparing the compound of the formula (VIII-1) as described above, wherein trifluoromethanesulfonic anhydride is added in a molar excess, preferably in a ratio of 1.5:1 based on the compound of the formula (III).

The present invention further provides a process for preparing the compound of the formula (VIII-1) as described above, wherein the compound of the formula (VII-1) is used in a molar ratio of 1:1 to 1.1:1 based on the compound of the formula (III).

The present invention further provides a process for preparing the compound of the formula (VIII-1) as described above, wherein the process takes place with exclusion of water, preferably under a protective gas atmosphere, more preferably while sparging with argon.

The present invention further provides a process for preparing the compound of the formula (VIII-1) as described above, wherein the process takes place with exclusion of water, preferably under a protective gas atmosphere, more preferably while sparging with argon.

### Example 5

Process Step 5

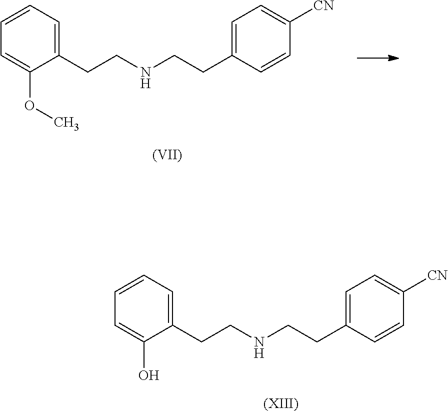

For preparation of 4-(2-{[2-(2-hydroxyphenyl)ethyl]amino}ethyl)benzonitrile of the formula (XIII) in process step 5 (Scheme 3), aluminium chloride is first stirred until dissolution with a suitable alkyl thiol, preferably n-dodecanethiol (dodecyl mercaptan), preferably in a molar ratio between 1:1 and 1:3, more preferably 1:1.8. At a temperature of 0 to 40° C., preferably 10 to 20° C., 4-(2-{[2-(2-methoxyphenyl)ethyl]amino}ethyl)benzonitrile of the formula (VII) is added and the mixture is preferably stirred at a temperature of 30 to 50° C., more preferably 40° C., for several hours.

It has been found that, surprisingly, preparation with the aid of aluminium chloride is advantageous since the compound of the formula (XIII) is insoluble as the aluminium complex (Al complex) in suitable solvents, for example DCM or toluene, and precipitates out. The solubility of the Al complex is dependent on the solvent; for instance, it is soluble in THF. This unforeseeable circumstance can advantageously be used for the purification of the reaction mixture in that the reaction product formed is isolated as the insoluble Al complex and washed with suitable solvents, preferably DCM or toluene, more preferably DCM. Subsequently, the complex can be dissolved in a suitable solvent, preferably THF, and the compound of the formula (XIII) can be released from the complex by adding a tartrate, preferably potassium sodium tartrate solution, in a molar excess based on the compound of the formula (VII). The release from the complex by addition of a tartrate can be performed repeatedly.

The compound of the formula (XIII) is preferably isolated after an aqueous basic workup. Suitable methods of aqueous basic workup are extractions that are known to the person skilled in the art and are capable of separating off by-products. For this purpose, for example, the solvent is changed to DCM, aqueous ammonia solution, preferably 27% aqueous ammonia solution, is added, the mixture is washed with water and the organic phase is concentrated to an oil.

The present invention further provides the compound of the formula (XIII)

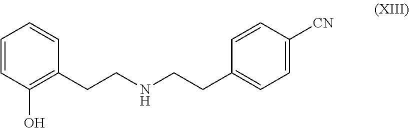 [SMILES: N#Cc1ccc(CCNCCc2ccccc2O)cc1]

and the salts, solvates and solvates of the salts thereof.

The present invention further provides a process for preparing the compound of the formula (XIII), characterized in that in a first step aluminium chloride is mixed with a suitable alkyl thiol, and in a second step is reacted with the compound of the formula (VII)

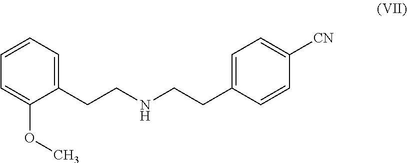 [SMILES: COc1ccccc1CCNCCc1ccc(C#N)cc1]

in the solvent dichloromethane or toluene.

The present invention further provides a process for preparing the compound of the formula (XIII) as described above, wherein the suitable alkyl thiol is n-dodecanethiol.

The present invention further provides a process for preparing the compound of the formula (XIII) as described above, wherein the solvent is toluene and/or dichloromethane, preferably dichloromethane.

The present invention further provides a process for preparing the compound of the formula (XIII) as described above, wherein the suitable alkyl thiol is added in a molar ratio of 1:1 to 1:3 based on the compound of the formula (VII), more preferably in a molar ratio of 1:1.8 based on the compound of the formula (VII).

The present invention further provides a process for preparing the compound of the formula (XIII) as described above, wherein the compound of the formula (VII) is added at a temperature of 0° C. to 40° C., preferably 10° C. to 20° C.

The present invention further provides a process for preparing the compound of the formula (XIII) as described above, wherein conversion in the second step is effected at a temperature of 30° C. to 50° C., more preferably 40° C.

The present invention further provides a process for preparing the compound of the formula (XIII) as described above, wherein the insoluble compound of the formula (XIII) formed is isolated and dissolved in tetrahydrofuran, and a tartrate solution is added.

### Example 6

Process steps 6A and 6B describe the preparation of 4-(2-{[2-(2-{[tert-butyl(dimethyl)silyl]oxy}phenyl)ethyl]amino}ethyl)benzonitrile of the formula (XIV).

Process Step 6A:

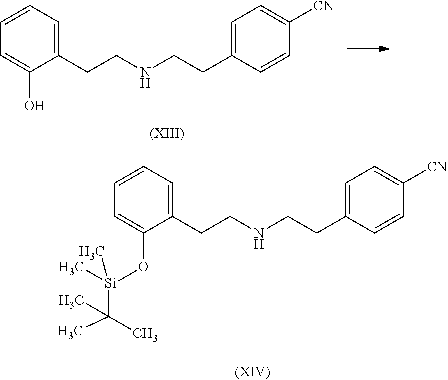

For preparation of 4-(2-{[2-(2-{[tert-butyl(dimethyl)silyl]oxy}phenyl)ethyl]amino}ethyl)benzonitrile of the formula (XIV) by process step 6A (Scheme 3), 4-(2-{[2-(2-hydroxyphenyl)ethyl]amino}ethyl)benzonitrile of the formula (XIII) is dissolved in a suitable solvent, for example an ether or a halohydrocarbon, preferably DCM. At a temperature of 0° C. to 40° C., preferably 20° C. to 35° C., the hydroxyl group of the compound of the formula (XIII) is protected with a silyl protecting group. Silyl protecting groups used may be silyl protecting groups known to those skilled in the art, for example trimethylsilyl (TMS), triethylsilyl (TES), triisopropylsilyl (TIPS), tert-butyldiphenylsilyl (TBDPS) or tert-butyldimethylsilyl (TBDMS); preference is given to tert-butyldimethylsilyl (TBDMS). For this purpose, the compound of the formula (XIII) is stirred with an appropriate silyl chloride, preferably tert-butyldimethylsilyl chloride, in the presence of an amine base, preferably imidazole, at a temperature of 0° C. to 40° C., preferably 20° C. to 35° C., until conversion is complete. The amine base is present in a molar ratio of 1:1 or in excess relative to the compound of the formula (XIII), preferably in a 1.5-fold molar excess.

Prior to the concentration, the reaction mixture can be purified by an aqueous basic purification known to the person skilled in the art. For this purpose, for example, an aqueous potassium carbonate solution is added to the reaction mixture, the organic phase is washed repeatedly with water, and the organic phase is dried with sodium sulfate.

An alternative preferred purification can be achieved by precipitating the compound of the formula (XIV) as the oxalic salt. For this purpose, after washing the reaction mixture with water, the solvent of the organic phase is changed to methanol and the mixture is heated to a temperature of 40° C. to 80° C., preferably 65° C. After addition of oxalic acid in excess based on the compound of the formula (XIV), the mixture is stirred at a temperature of 40° C. to 65° C., preferably 50° C. to 55° C., and then cooled to a temperature of 0° C. to 20° C., preferably 5° C. to 10° C. The precipitated solids are separated off and suspended and stirred in a mixture of water and an inert solvent that shows phase separation with water, for example DCM, toluene or an ether, preferably DCM. After the pH has been adjusted to 10.5 to 12.5 by means of a suitable base, for example and with preference sodium hydroxide solution, the phases are separated and the organic phase is concentrated.

Concentration is effected at a temperature of 25° C. to 70° C., preferably 30° C. to 50° C., more preferably 35° C., preferably under reduced atmospheric pressure, and the compound of the formula (XIV) is obtained as an oil.

The present invention further provides the compound of the formula (XIV)

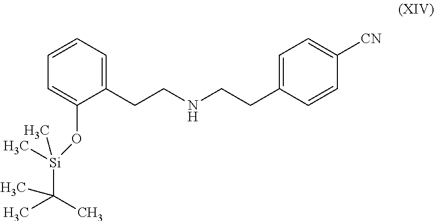 [SMILES: CC(C)(C)[Si](C)(C)Oc1ccccc1CCNCCc1ccc(C#N)cc1]

and the salts, solvates and solvates of the salts thereof.

The present invention further provides the compound of the formula (XIV-1)

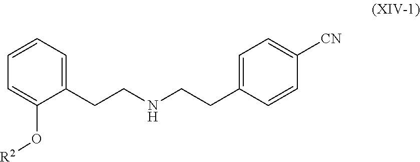

where

R² is a silyl protecting group

and the salts, solvates and solvates of the salts thereof.

The present invention further provides a process for preparing the compound of the formula (XIV-1)

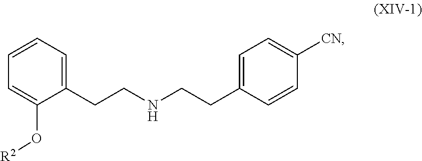

where

R² is a silyl protecting group,

characterized in that the compound of the formula (XIII)

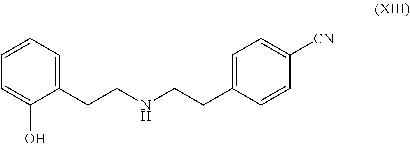 [SMILES: N#Cc1ccc(CCNCCc2ccccc2O)cc1]

is reacted with the appropriate silyl chloride in the presence of an amine base.

In the context of the present invention, a “silyl protecting group” is a silyl protecting group which is known to the person skilled in the art and is capable of converting a reactive functional group to an unreactive form by means of an organosilicon compound. Preference is given to using trimethylsilyl (TMS), triethylsilyl (TES), triisopropylsilyl (TIPS), tert-butyldiphenylsilyl (TBDPS) or tert-butyldimethylsilyl (TBDMS), particular preference to using tert-butyldimethylsilyl (TBDMS).

In the context of the present invention, the appropriate silyl chloride is that silyl chloride which is used for preparation of the respective silyl protecting group.

The present invention further provides a process for preparing the compound of the formula (XIV-1), wherein the amine base is imidazole.

The present invention further provides a process for preparing the compound of the formula (XIV-1), where R² is selected from a group comprising trimethylsilyl, triethylsilyl, triisopropylsilyl, tert-butyldiphenylsilyl and tert-butyldimethylsilyl.

The present invention further provides a process for preparing the compound of the formula (XIV-1), where R² is tert-butyldimethylsilyl.

The present invention further provides a process for preparing the compound of the formula (XIV-1), wherein the appropriate silyl chloride is selected from a group comprising trimethylsilyl chloride, triethylsilyl chloride, triisopropylsilyl chloride, tert-butyldiphenylsilyl chloride and tert-butyldimethylsilyl chloride.

The present invention further provides a process for preparing the compound of the formula (XIV-1), wherein the appropriate silyl chloride is tert-butyldimethylsilyl chloride.

The present invention further provides a process for preparing the compound of the formula (XIV-1), wherein the amine base is present in a molar ratio of 1.5:1 or in excess, based on the compound of the formula (XIII).

Process Step 6B:

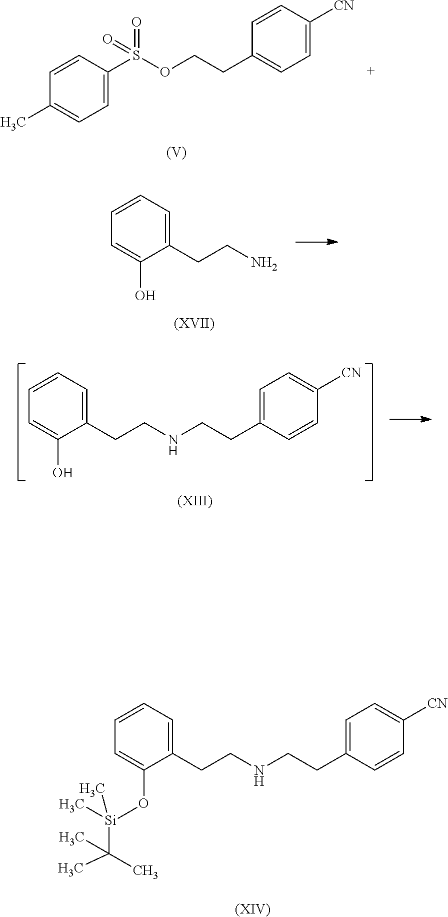

Alternatively, the compound of the formula (XIV) can be prepared in process step 6B (Scheme 4) from 2-(4-cyanophenyl)ethyl 4-methylbenzenesulfonate of the formula (V) and 2-(2-aminoethyl)phenol of the formula (XVII). For this purpose, the compound of the formula (V) is dissolved in a suitable solvent, for example an ether, preferably DCM or THF, more preferably THF, and 2-(2-aminoethyl)phenol of the formula (XVII), preferably in a ratio of 2:1 or higher based on the compound of the formula (V), and triethylamine, preferably in a ratio of 3:1 or higher based on the compound of the formula (V), are added. The reaction mixture is heated for several hours, preferably 20 to 60 hours, more preferably 46 hours, preferably at a temperature corresponding to the boiling temperature of the reaction mixture. If DCM has not been used as solvent, the solvent is preferably changed to DCM. This can be effected, for example, by removing the original solvent under reduced pressure and at a temperature of 60° C. or less, and then adding DCM. The solution can then be washed by known methods, for example with preference by one or more washes with sodium bicarbonate and optionally further concentrated, for example with preference at temperatures of 45° C. or less.

Imidazole is added to the resulting solution, preferably in a ratio of 2:1 to 5:1, preferably 3:1, based on the compound of the formula (V), and stirred at a temperature of 20 to 35° C., more preferably room temperature, until conversion is complete.

This may be followed by an aqueous basic purification known to the person skilled in the art. The following process is preferably envisaged for this purpose: The reaction mixture is washed once or more than once with water, and the solvent is changed to methanol. Oxalic acid is added at a temperature of 40 to 70° C., preferably 50 to 55° C., and the mixture is stirred. After cooling to a temperature of 0 to 20° C., preferably 5 to 10° C., the solids are isolated and washed with methanol. The residue is suspended in a mixture of DCM or toluene and water, preferably DCM and water, preferably in a volume ratio of 1:1, and, at a temperature of 15 to 40° C., preferably 25 to 35° C., admixed with a concentrated base, preferably sodium hydroxide solution, more preferably 45% sodium hydroxide solution, and a pH between 10.5 and 12.5 is attained. After addition of water, the organic phase was isolated and preferably concentrated under reduced atmospheric pressure. The compound of the formula (XIV) is obtained as an oil.

One advantage of this process is that the alkylation of 2-(2-aminoethyl)phenol of the formula (V), without prior protection of the hydroxyl function of the phenol (which can enter into alkylation reactions under basic conditions), can give the desired monoalkylation product of the primary amine.

The present invention further provides a process for preparing the compound of the formula (XIV-1)

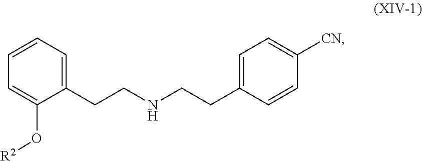

where

R² is a silyl protecting group,

characterized in that the compounds of the formulae (XVI) and (V)

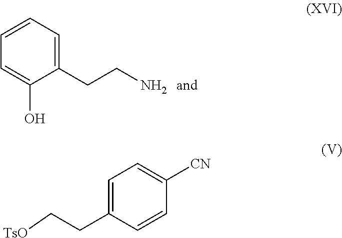 [SMILES: NCCc1ccccc1O ; Cc1ccc(S(=O)(=O)OCCc2ccc(C#N)cc2)cc1]

in a first step are coupled in the presence of an amine base, for example triethylamine, and the reaction product in a second step is reacted with the appropriate silyl chloride, likewise in the presence of an amine base.

The present invention further provides a process for preparing the compound of the formula (XIV-1) as described above, wherein the amine base in the first step is triethylamine.

The present invention further provides a process for preparing the compound of the formula (XIV-1) as described above, wherein the first step takes place in a suitable ether as solvent.

The present invention further provides a process for preparing the compound of the formula (XIV-1) as described above, wherein the suitable ether is dichloromethane or tetrahydrofuran.

The present invention further provides a process for preparing the compound of the formula (XIV-1) as described above, wherein the compound of the formula (XVI) is used in a molar ratio of 2:1 or higher, based on the compound of the formula (V).

The present invention further provides a process for preparing the compound of the formula (XIV-1) as described above, wherein triethylamine is used in a molar ratio of 3:1 or higher, based on the compound of the formula (V).

The present invention further provides a process for preparing the compound of the formula (XIV-1) as described above, wherein the conversion in the first step is effected for several hours, preferably 20 to 60 hours, more preferably 46 hours, at boiling temperature.

The present invention further provides a process for preparing the compound of the formula (XIV-1) as described above, wherein the amine base in the second step is imidazole.

The present invention further provides a process for preparing the compound of the formula (XIV-1) as described above, wherein the amine base in the second step is used in a molar ratio of 2:1 to 5:1, preferably 3:1, based on the compound of the formula (V).

The present invention further provides a process for preparing the compound of the formula (XIV-1) as described above, wherein the second step takes place at a temperature of 20° C. to 35° C.

### Example 7

Process Step 7

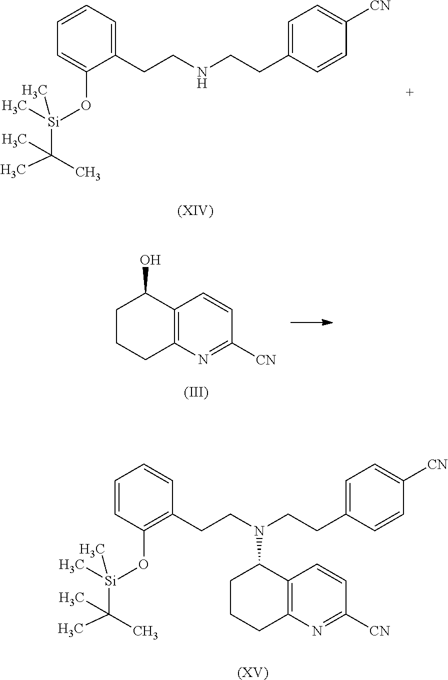

For preparation of (5S)-5-{[2-(2-{[tert-butyl(dimethyl)silyl]oxy}phenyl)ethyl][2-(4-cyanophenyl)ethyl]amino}-5,6,7,8-tetrahydroquinoline-2-carbonitrile of the formula (XV) in process step 7 (Scheme 3), (5R)-5-hydroxy-5,6,7,8-tetrahydroquinoline-2-carbonitrile of the formula (III) is added to a suitable solvent. Suitable solvents are those that are liquid at the reaction temperatures, for example THF or DCM; preference is given to using DCM. A suitable base is added to the solution. Suitable bases are sterically hindered secondary amines or 2,6-disubstituted pyridines. Suitable sterically hindered secondary amines are, for example, diisopropylamine, 2,5-dimethylpiperidine or 2,2,5,5-tetramethylpiperidine, preferably diisopropylamine. Particular preference is given to adding an excess of diisopropylamine, more preferably 3 eq. of diisopropylamine, based on the compound of the formula (X). It is surprisingly possible with these compounds to achieve better yields compared to sterically unhindered or tertiary amines. It was especially surprising that the most advantageous yields are achieved by the use of diisopropylamine.

The reaction mixture is cooled to a temperature between −90° C. and −50° C., preferably −78° C. and −65° C., and trifluoromethanesulfonic anhydride is added, preferably in excess, more preferably 1.5 eq. based on the compound of the formula (III), and the mixture is stirred. While maintaining the temperature range mentioned, 4-(2-{[2-(2-{[tert-butyl(dimethyl)silyl]oxy}phenyl)ethyl]amino}ethyl)benzonitrile of the formula (XIV) is added dissolved in DCM, preferably in equimolar amounts based on the compound of the formula (III), more preferably 1.0 eq. to 1.1. eq., based on the compound of the formula (III), and the mixture is stirred until conversion is complete. Subsequently, the reaction mixture is warmed to a temperature of 10 to 30° C., preferably 20° C.

Prior to the concentration, the reaction mixture can be purified by an aqueous acidic purification known to the person skilled in the art. For this purpose, the reaction mixture is acidified with a mineral acid, preferably phosphoric acid or hydrochloric acid, more preferably hydrochloric acid, and optionally washed with water, and the organic phase is isolated.

Concentration is effected at a temperature of 30° C. to 80° C., preferably 30° C. to 60° C., more preferably 40° C., preferably under reduced atmospheric pressure, and the compound of the formula (XV) is obtained as an oil.

Optionally, the oil obtained can be filtered through silica gel. For this purpose, the oil is dissolved in a suitable solvent, preferably DCM, filtered through silica gel and diluted with a suitable solvent, preferably a solvent mixture of ethyl acetate and n-hexane in a ratio of 1:2 (ethyl acetate:n-hexane). Subsequently, the product solution is concentrated again under the conditions described above.

The present invention further provides the compound of the formula (XV)

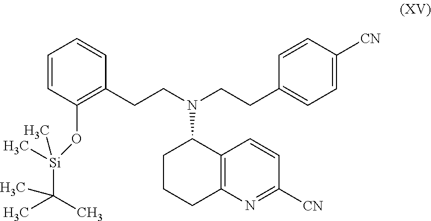 [SMILES: CC(C)(C)[Si](C)(C)Oc1ccccc1CCN(CCc1ccc(C#N)cc1)[C@H]1CCCc2nc(C#N)ccc21]

and the salts, solvates and solvates of the salts thereof.

The present invention further provides the compound of the formula (XV-1)

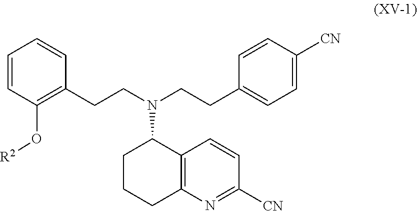

where

R² is a silyl protecting group,

and the salts, solvates and solvates of the salts thereof.

The present invention further provides a process for preparing the compound of the formula (XV-1)

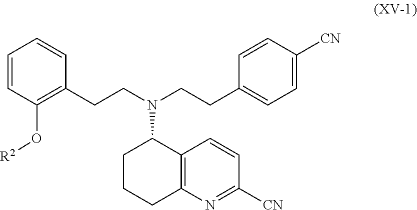

where

R² is a silyl protecting group,

characterized in that, at a temperature of −90° C. to −50° C. in a first step, it is added to the compound of the formula (III)

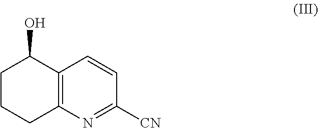 [SMILES: N#Cc1ccc2c(n1)CCC[C@H]2O]

in the presence of a base selected from a list consisting of sterically hindered secondary amines and 2,6-disubstituted pyridines and trifluoromethanesulfonic anhydride, and in a second step is reacted with the compound of the formula (XVI-1)

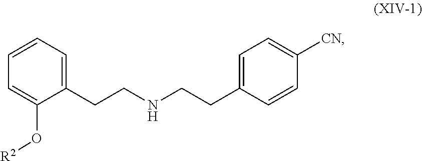

where

R² is a silyl protecting group.

The present invention further provides a process for preparing the compound of the formula (XV-1) as described above, wherein the sterically hindered secondary amine is selected from a list comprising diisopropylamine, 2,5-dimethylpiperidine and 2,2,5,5-tetramethylpiperidine.

The present invention further provides a process for preparing the compound of the formula (XV-1) as described above, wherein the base is diisopropylamine.

The present invention further provides a process for preparing the compound of the formula (XV-1) as described above, wherein the temperature is −78° C. to −65° C., preferably −76° C.

The present invention further provides a process for preparing the compound of the formula (XV-1) as described above, wherein the compound of the formula (III) has been dissolved in tetrahydrofuran or dichloromethane, preferably dichloromethane.

The present invention further provides a process for preparing the compound of the formula (XV-1) as described above, wherein trifluoromethanesulfonic anhydride is added in a molar excess, preferably in a ratio of 1.5:1 based on the compound of the formula (III).

The present invention further provides a process for preparing the compound of the formula (XV-1) as described above, wherein the base is in a molar excess, preferably in a ratio of 3:1 based on the compound of the formula (III).

The present invention further provides a process for preparing the compound of the formula (XV-1) as described above, wherein the compound of the formula (XIV-1) is used in a molar ratio of 1:1 to 1.1:1, based on the compound of the formula (III).

The present invention further provides a process for preparing the compound of the formula (XV-1) as described above, wherein the process takes place with exclusion of water, preferably under a protective gas atmosphere, more preferably while sparging with argon.

### Example 8

Process step 8

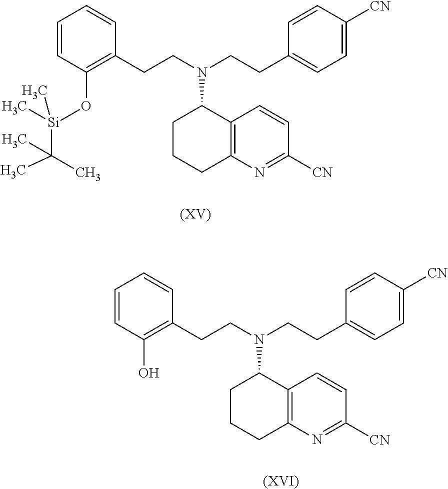

For preparation of (5S)-5-{[2-(4-cyanophenyl)ethyl][2-(2-hydroxyphenyl)ethyl]amino}-5,6,7,8-tetrahydroquinoline-2-carbonitrile of the formula (XVI) in process step 8 (Scheme 3), (5S)-5-{[2-(2-{[tert-butyl(dimethyl)silyl]oxy}phenyl)ethyl][2-(4-cyanophenyl)ethyl]amino}-5,6,7,8-tetrahydroquinoline-2-carbonitrile of the formula (XV) in a suitable alcohol, preferably methanol, is admixed with highly concentrated hydrochloric acid, preferably 37% or 25% hydrochloric acid, at a temperature of 10° C. to 40° C., preferably 25° C., until conversion is complete.

After neutralization with aqueous ammonia solution, preferably 30% ammonia solution, the compound of the formula (VI) can be extracted in solid form. These solids can be stirred in a mixture of water and dichloromethane, and the organic phase can be washed with water and concentrated.

The solids obtained can also be dissolved in a mixture of methanol and water at reflux temperature and, by cooling to room temperature, can surprisingly be obtained with relatively high enantiomeric purity without addition of chiral reagents. This is particularly advantageous for the preparation of enantiomerically pure active ingredient.

The present invention further provides the compound of the formula (XVI)

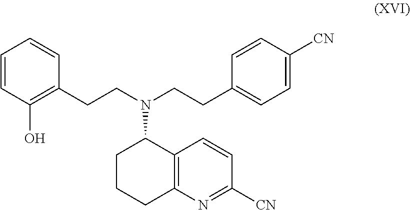 [SMILES: N#Cc1ccc(CCN(CCc2ccccc2O)[C@H]2CCCc3nc(C#N)ccc32)cc1]

and the salts, solvates and solvates of the salts thereof.

The present invention further provides a process for preparing the compound of the formula (XVI), characterized in that the compound of the formula (XV-1)

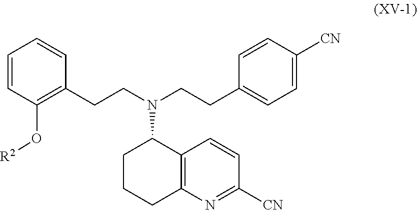

where

R² is a silyl protecting group.

is reacted with a mineral acid.

The present invention further provides a process for preparing the compound of the formula (XVI) as described above, wherein the mineral acid is hydrochloric acid, preferably 25% hydrochloric acid.

The present invention further provides a process for preparing the compound of the formula (XVI) as described above, wherein the conversion takes place at a temperature of 10° C. to 40° C., preferably 25° C.

The present invention further provides a process for preparing the compound of the formula (XVI) as described above, wherein the conversion takes place in methanol.

The present invention further provides a process for preparing the compound of the formula (XVI) as described above, wherein, after the reaction, the mixture is admixed with ammonia solution, preferably 30% ammonia solution, and the compound of the formula (VI) is extracted in solid form.

### Example 9

Process Step 9A:

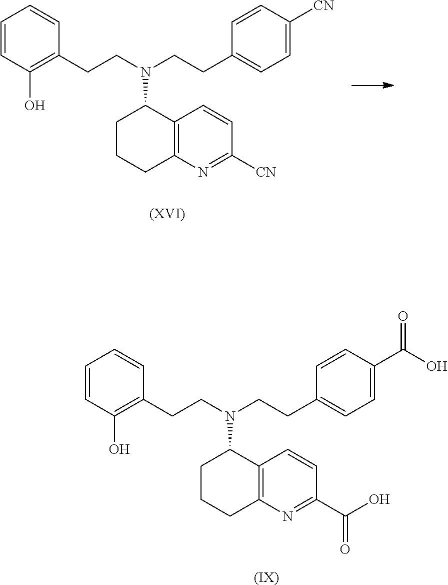

For preparation of (5S)-5-{[2-(4-carboxyphenyl)ethyl][2-(2-hydroxyphenyl)ethyl]amino}-5,6,7,8-tetrahydroquinoline-2-carboxylic acid of the formula (IX) in process step 9A (Scheme 3), (5S)-5-{[2-(4-cyanophenyl)ethyl][2-(2-hydroxyphenyl)ethyl]amino}-5,6,7,8-tetrahydroquinoline-2-carbonitrile of the formula (XVI) is suspended in highly concentrated hydrochloric acid, preferably 25% hydrochloric acid, at a temperature of 90° C. to 110° C., preferably 103° C., until conversion is complete. The reaction product can be used directly in the next stage.

Alternatively, the reaction product is first cooled down to a temperature of 15° C. to 50° C., preferably 40° C., the suspension is filtered and the filtrate is used for use in the next stage.

The present invention further provides the compound of the formula (IX)

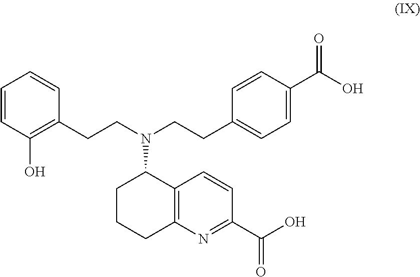 [SMILES: O=C(O)c1ccc(CCN(CCc2ccccc2O)[C@H]2CCCc3nc(C(=O)O)ccc32)cc1]

and the salts, solvates and solvates of the salts thereof.

The present invention further provides a process for preparing the compound of the formula (IX), characterized in that the compound of the formula (XVI)

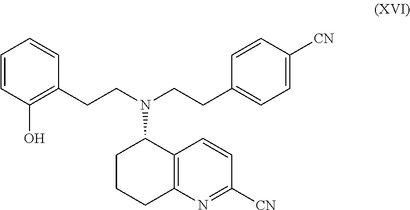 [SMILES: N#Cc1ccc(CCN(CCc2ccccc2O)[C@H]2CCCc3nc(C#N)ccc32)cc1]

is reacted with a mineral acid.

The present invention further provides a process for preparing the compound of the formula (IX) as described above, wherein the mineral acid is hydrochloric acid, preferably 25% hydrochloric acid.

The present invention further provides a process for preparing the compound of the formula (IX) as described above, wherein the conversion takes place at a temperature of 90° C. to 110° C., preferably 103° C.

Process Step 9B:

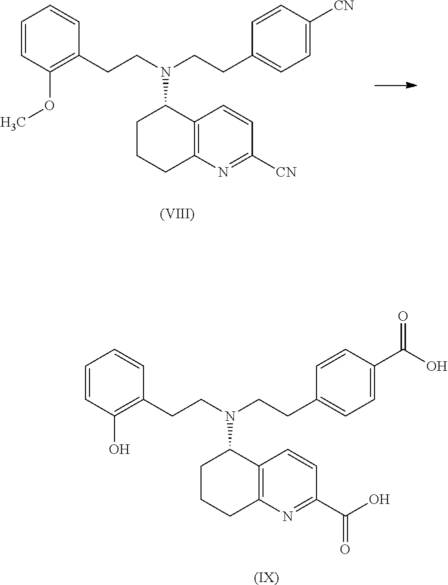

In an alternative process step 9B (Scheme 2), the compound of the formula (IX) can be prepared from (5S)-5-{[2-(4-cyanophenyl)ethyl][2-(2-methoxyphenyl)ethyl]amino}-5,6,7,8-tetrahydroquinoline-2-carbonitrile of the formula (VIII). For this purpose, the compound of the formula (VIII) is suspended with highly concentrated hydrobromic acid, preferably 48% hydrobromic acid, and stirred at a temperature of 90° C. to 110° C., preferably 108° C., until conversion is complete. Subsequently, the reaction product is first cooled down to a temperature of 15° C. to 40° C., preferably 25° C., and washed with DCM, and aqueous phase is used for use in the next stage.

Process 9B gives rise to toxic methyl bromide; therefore, the gases formed in the reaction must be collected by a gas scrubber. In addition, hydrobromic acid used as reactant has highly corrosive properties.

The present invention further provides a process for preparing the compound of the formula (IX), characterized in that the compound of the formula (VIII)

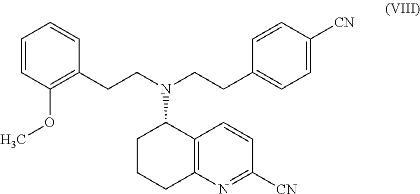 [SMILES: COc1ccccc1CCN(CCc1ccc(C#N)cc1)[C@H]1CCCc2nc(C#N)ccc21]

is reacted with hydrobromic acid at a temperature of 90° C. to 110° C.

The present invention further provides a process for preparing the compound of the formula (IX) as described above, wherein 48% hydrobromic acid is used.

The present invention further provides a process for preparing the compound of the formula (IX) as described above, wherein the conversion takes place at a temperature of 108° C.

### Example 10

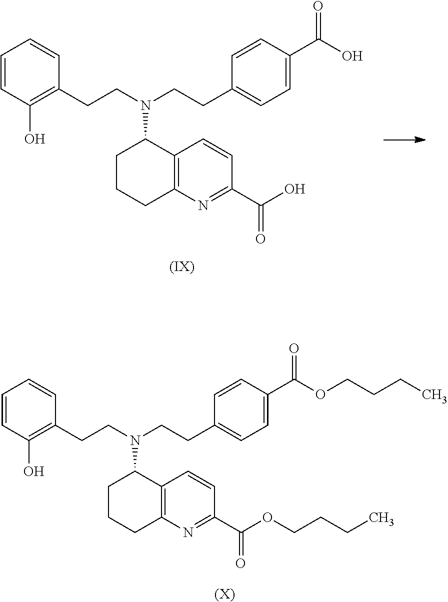

Process Step 10A:

For preparation of butyl (5S)-5-({2-[4-(butoxycarbonyl)phenyl]ethyl}[2-(2-hydroxyphenyl)ethyl]amino)-5,6,7,8-tetrahydroquinoline-2-carboxylate of the formula (X) in process step 10A (Schemes 2 and 3), (5S)-5-{[2-(4-carboxyphenyl)ethyl][2-(2-hydroxyphenyl)ethyl]amino}-5,6,7,8-tetrahydroquinoline-2-carboxylic acid of the formula (IX) is heated to boiling in a mineral acid, preferably hydrochloric acid, and a suitable alcohol, for example butanol, preferably n-butanol, as solvent and stirred until conversion is complete. Any aqueous solvent components that are present by virtue of the precursor, for example, and are formed in the reaction are removed. This can be effected, for example, by distillation with continuous addition of the organic solvent until the boiling temperature of the organic solvent has been attained. The steps described are preferably effected under reduced atmospheric pressure. This is followed by cooling down to a temperature of 10° C. to 30° C., preferably 22° C., and performance of an aqueous basic purification. Optionally, the cooling is followed by filtration, and performance of the aqueous basic purification with the filtrate.

Aqueous basic purification procedures are known to the person skilled in the art; for the aqueous basic purification, preference is given to adding ethyl acetate and an aqueous base, preferably ammonia solution or potassium carbonate and water, stirring, and removing and discarding the aqueous phase. In a second step, preference is given to adding water and sodium chloride to the remaining organic phase, stirring, and removing and discarding the aqueous phase. In a third step, preference is given to adding water to the remaining organic phase, stirring, and removing and discarding the aqueous phase. In a last step, the remaining organic phase is concentrated at a temperature of 30° C. to 80° C., preferably 40° C. to 70° C., more preferably 55° C., preferably under reduced atmospheric pressure, and the compound of the formula (X) is obtained as an oil.

Optionally, the oil obtained is dissolved in DCM and methanol and filtered with silica gel, and the filtrate obtained is concentrated again under the conditions described above to give an oil.

One advantage of this process is that water present or formed in the reaction can be removed very effectively from the reaction mixture by azeotropic distillation, and hence the reaction time before full conversion is attained can be shortened. Butanol is notable here, compared to other solvents, in that it removes considerably more water from the reaction mixture based on the amount of solvent distilled off compared to other solvents, for example acetonitrile. This has an advantageous effect on the distillation time. On the industrial scale, the shorter distillation time results in lower operating costs and apparatus occupation times and lower energy costs. Furthermore, the solvent which is used for the azeotropic distillation is simultaneously the reagent for formation of the butyl ester, which makes it unnecessary to use a further solvent. A further advantage of the process is that the end of the reaction, on attainment of full conversion, is indicated without further analytical studies by the attainment of the internal temperature at the boiling point of butanol under the chosen distillation conditions (distillation pressure). This is a considerable advantage particularly on an industrial scale.

The present invention further provides the compound of the formula (X)

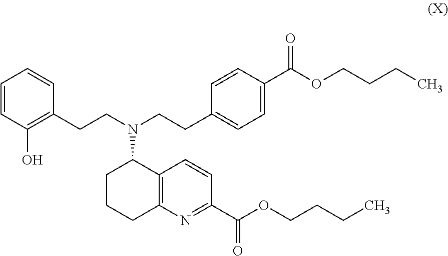 [SMILES: CCCCOC(=O)c1ccc(CCN(CCc2ccccc2O)[C@H]2CCCc3nc(C(=O)OCCCC)ccc32)cc1]

and the salts, solvates and solvates of the salts thereof.

The present invention further provides a process for preparing the compound of the formula (X), characterized in that the compound of the formula (IX)

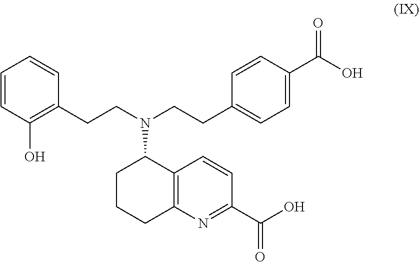 [SMILES: O=C(O)c1ccc(CCN(CCc2ccccc2O)[C@H]2CCCc3nc(C(=O)O)ccc32)cc1]

is reacted with n-butanol in the presence of a mineral acid.

The present invention further provides a process for preparing the compound of the formula (X) as described above, wherein n-butanol is used.

The present invention further provides a process for preparing the compound of the formula (X) as described above, wherein the mineral acid is hydrochloric acid.

The present invention further provides a process for preparing the compound of the formula (X) as described above, wherein the conversion takes place at boiling temperature.

Process Step 10B:

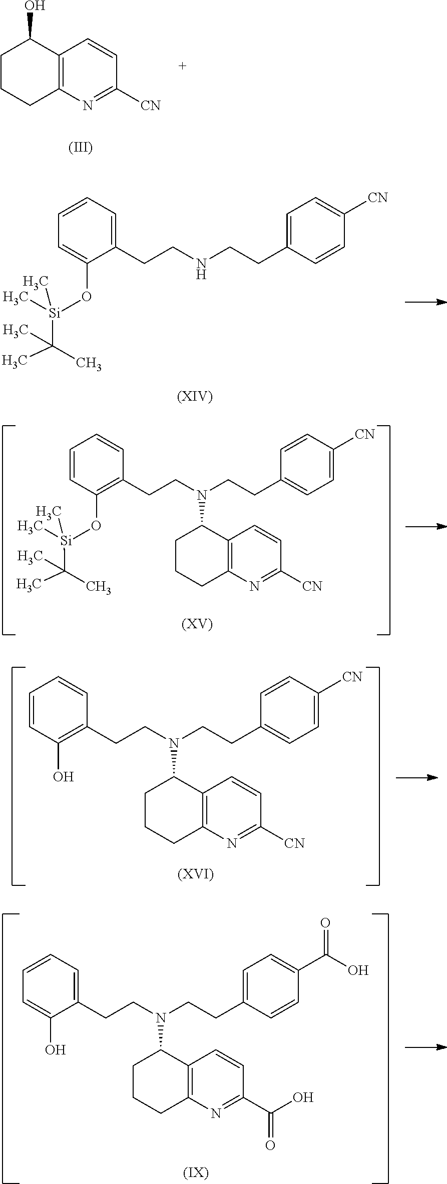

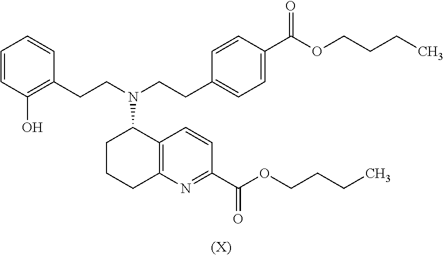

Alternatively, butyl (5S)-5-({2-[4-(butoxycarbonyl)phenyl]ethyl}[2-(2-hydroxyphenyl)ethyl]amino)-5,6,7,8-tetrahydroquinoline-2-carboxylate of the formula (X) can be obtained from the compounds of the formula (III) and formula (XIV) without isolation of intermediates (process step 10B—Scheme 4). For this purpose, process steps 7, 8, 9A and 10A are performed successively, and the respective products from the process steps are obtained as oils and used directly in the respective next stage.

The present invention further provides a process for preparing the compound of the formula (X)

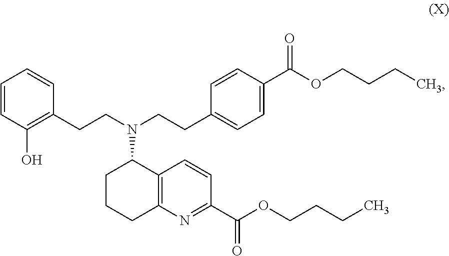 [SMILES: CCCCOC(=O)c1ccc(CCN(CCc2ccccc2O)[C@H]2CCCc3nc(C(=O)OCCCC)ccc32)cc1]

characterized in that, in a first step at a temperature of −90° C. to −50° C., it is added to the compound of the formula (III)

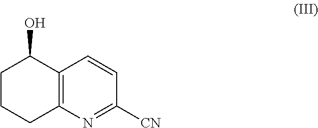 [SMILES: N#Cc1ccc2c(n1)CCC[C@H]2O]

in the presence of a base selected from a list consisting of sterically hindered secondary amines and 2,6-disubstituted pyridines, trifluoromethanesulfonic anhydride, and in a second step is reacted at a temperature of −90° C. to −50° C. with the compound of the formula (XIV-1)

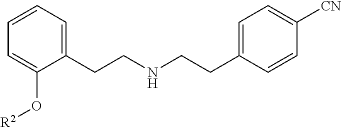

where

R² is a silyl protecting group,

in a third step the reaction product is reacted with hydrochloric acid, and in a fourth step the reaction product is reacted with butanol in the presence of a mineral acid.

The present invention further provides a process for preparing the compound of the formula (X) as described above, wherein the sterically hindered secondary amine is selected from a list comprising diisopropylamine, 2,5-dimethylpiperidine and 2,2,5,5-tetramethylpiperidine.

The present invention further provides a process for preparing the compound of the formula (X) as described above, wherein the base is diisopropylamine.

The present invention further provides a process for preparing the compound of the formula (X) as described above, wherein the temperature in the first and second steps is −78° C. to −65° C., preferably −76° C.

The present invention further provides a process for preparing the compound of the formula (X) as described above, wherein the compound of the formula (III) has been dissolved in tetrahydrofuran or dichloromethane, preferably dichloromethane.

The present invention further provides a process for preparing the compound of the formula (X) as described above, wherein trifluoromethanesulfonic anhydride is added in a molar excess, preferably in a ratio of 1.5:1 based on the compound of the formula (III).

The present invention further provides a process for preparing the compound of the formula (X) as described above, wherein the base is in a molar excess, preferably in a ratio of 3:1 based on the compound of the formula (III).

The present invention further provides a process for preparing the compound of the formula (X) as described above, wherein the compound of the formula (XIV-1) is used in a molar ratio of 1:1 to 1.1:1, based on the compound of the formula (III).

The present invention further provides a process for preparing the compound of the formula (X) as described above, wherein the process takes place with exclusion of water, preferably under a protective gas atmosphere, more preferably while sparging with argon.

The present invention further provides a process for preparing the compound of the formula (X) as described above, wherein the butanol used is n-butanol.

The present invention further provides a process for preparing the compound of the formula (X) as described above, wherein the mineral acid is hydrochloric acid.

The present invention further provides a process for preparing the compound of the formula (X) as described above, wherein the conversion in the third step takes place at a temperature of 90° C. to 110° C., preferably 103° C., and the conversion in the fourth step takes place at boiling temperature.

### Example 11

Process Step 11

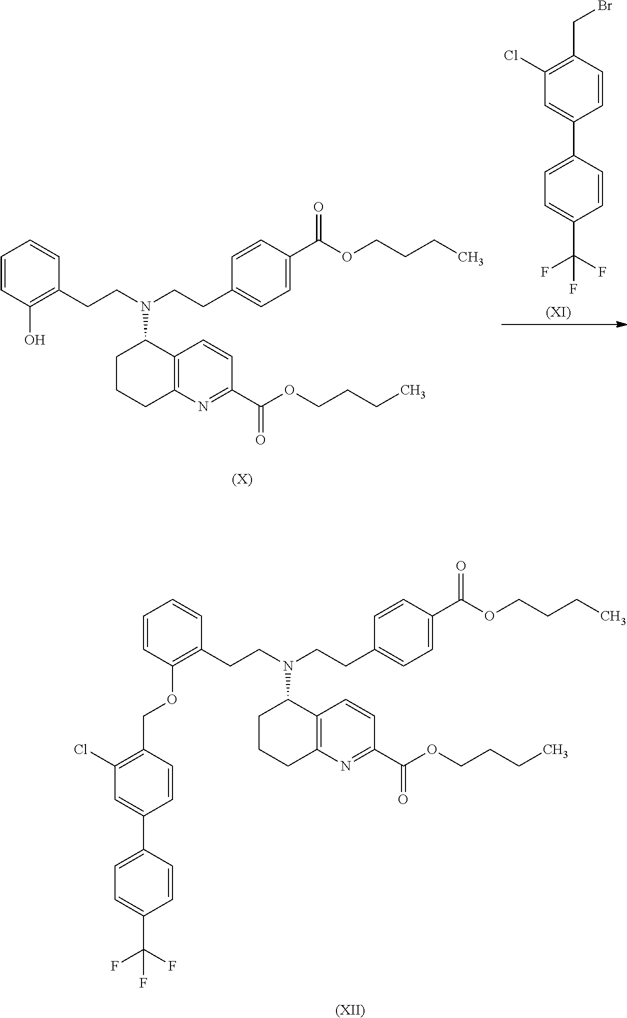

For preparation of the compound of the formula (XII), butyl (5S)-5-({2-[4-(butoxycarbonyl)phenyl]ethyl}[2-(2-hydroxyphenyl)ethyl]amino)-5,6,7,8-tetrahydroquinoline-2-carboxylate of the formula (X) (process step 11—Scheme 4) is dissolved in an inert polar solvent, for example suitable ethers, acetone or acetonitrile, preferably acetonitrile, preferably at a temperature of 10° C. to 40° C., preferably 25° C. Preference is then given to distilling at a temperature of 40° C. to 60° C. and reduced atmospheric pressure, preferably at 80 mbar to 120 mbar, more preferably 120 mbar, and adding acetonitrile. This step can be repeated.

4-(Bromomethyl)-3-chloro-4′-(trifluoromethyl)[biphenyl] of the formula (XI) is added to the solution, preferably in an amount of 1 eq to 2 eq, more preferably 1.2 eq, based on the compound of the formula (X). An additive is added to the solution, selected from a list comprising alkali metal carbonates, for example sodium carbonate, potassium carbonate or caesium carbonate, or alkali metal hydroxides, for example potassium hydroxide or sodium hydroxide, or tetraalkylammonium carbonates, for example tetramethyl-, tetraethyl-, tetrapropyl- or tetrabutylammonium carbonate, benzyltrimethyl-, benzyltriethyl-, benzyltripropyl- or benzyltributylammonium carbonate; preference is given to using caesium carbonate. The additive is added in a molar excess, preferably 2 eq to 4 eq, more preferably 2 eq, based on the compound of the formula (X). The mixture is stirred until conversion to the compound of the formula (XII) is complete. It is possible with preference to add a further amount of the additive, preferably caesium carbonate, to the reaction mixture, and to stir again. The resultant suspension is filtered. Before the filter residue is discarded, it is preferably washed with acetonitrile.

Alternatively, the compound of the formula (XII) can be isolated as an oil. For isolation of the oil, the filtrate is concentrated at a temperature of 15° C. to 60° C., preferably 30° C. to 50° C., more preferably 40° C., to give an oil. The concentration preferably takes place under reduced atmospheric pressure.

The present invention further provides the compound of the formula (XII)

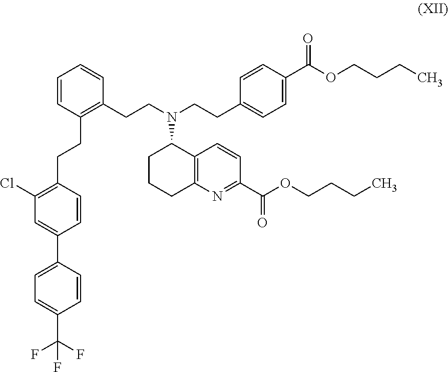 [SMILES: CCCCOC(=O)c1ccc(CCN(CCc2ccccc2CCc2ccc(-c3ccc(C(F)(F)F)cc3)cc2Cl)[C@H]2CCCc3nc(C(=O)OCCCC)ccc32)cc1]

and the salts, solvates and solvates of the salts thereof.

The present invention further provides a process for preparing the compound of the formula (XII-1)

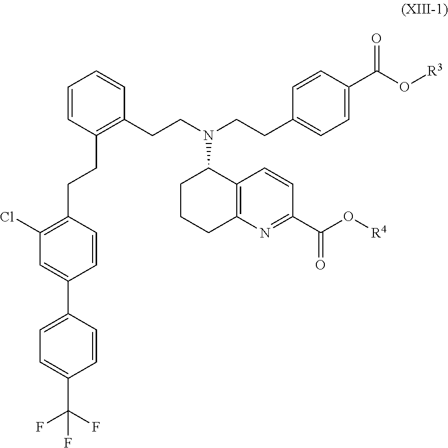

where

R³ and R⁴ are independently C₁-C₄-alkyl,

characterized in that the compound of the formula (X-1)

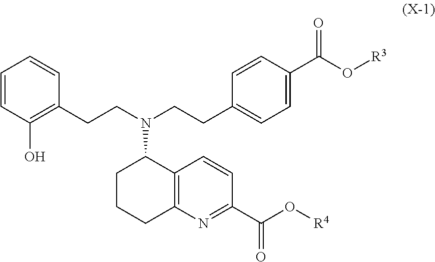

where

R³ and R⁴ are independently C₁-C₄-alkyl,

in the presence of an alkali metal carbonate, alkali metal hydroxide or tetraalkylammonium carbonate is reacted with the compound of the formula (XI)

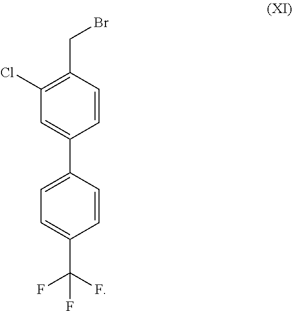 [SMILES: FC(F)(F)c1ccc(-c2ccc(CBr)c(Cl)c2)cc1]

The present invention further provides a process for preparing the compound of the formula (XII)

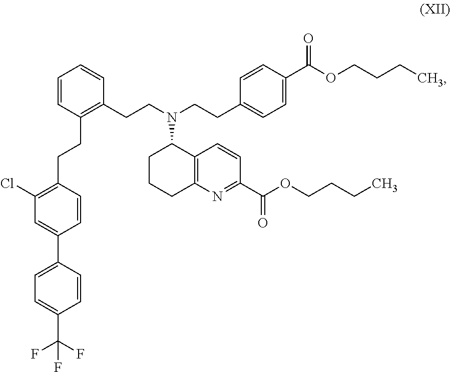 [SMILES: CCCCOC(=O)c1ccc(CCN(CCc2ccccc2CCc2ccc(-c3ccc(C(F)(F)F)cc3)cc2Cl)[C@H]2CCCc3nc(C(=O)OCCCC)ccc32)cc1]

characterized in that the compound of the formula (X)

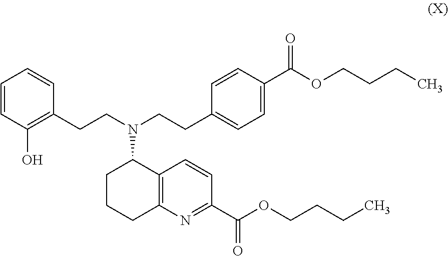 [SMILES: CCCCOC(=O)c1ccc(CCN(CCc2ccccc2O)[C@H]2CCCc3nc(C(=O)OCCCC)ccc32)cc1]

in the presence of an alkali metal carbonate, alkali metal hydroxide or tetraalkylammonium carbonate is reacted with the compound of the formula (XI)

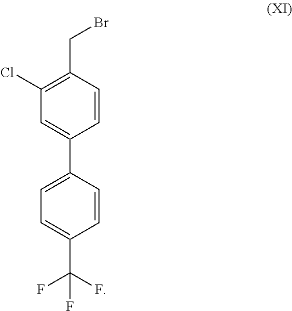 [SMILES: FC(F)(F)c1ccc(-c2ccc(CBr)c(Cl)c2)cc1]

The present invention further provides a process for preparing the compound of the formula (XII-1) as described above, wherein a suitable ether, acetone or acetonitrile, preferably acetonitrile, is used as solvent.

The present invention further provides a process for preparing the compound of the formula (XII-1) as described above, wherein an alkali metal carbonate selected from a list comprising sodium carbonate, potassium carbonate and caesium carbonate is used, preferably caesium carbonate.

The present invention further provides a process for preparing the compound of the formula (XII-1) as described above, wherein an alkali metal hydroxide selected from a list comprising sodium hydroxide and potassium hydroxide is used.

The present invention further provides a process for preparing the compound of the formula (XII-1) as described above, wherein a tetraalkylammonium carbonate is used.

The present invention further provides a process for preparing the compound of the formula (XII-1) as described above, wherein the alkali metal carbonate, alkali metal hydroxide or tetraalkylammonium carbonate is used in a molar excess, preferably in a molar ratio of 2:1 to 4:1 based on the compound of the formula (X), more preferably in a molar ratio of 2:1 based on the compound of the formula (X).

The present invention further provides a process for preparing the compound of the formula (XII-1) as described above, wherein the compound of the formula (XI) is used preferably in a molar ratio of 1:1 to 2:1 based on the compound of the formula (X), more preferably in a molar ratio of 1.2:1 based on the compound of the formula (X).

The compound of the formula (I) can be prepared from the compound of the formula (XII) or (XII-A) by an ester hydrolysis method known to those skilled in the art. By way of example, an ester hydrolysis can be effected analogously to the method described in Example 23 of WO 2014/012934.

### Experimental Section

### Abbreviations and Acronyms

abs. absolute
acac acetylacetonato
BINAP (2,2′-bis(diphenylphosphino)-1,1′-binaphthyl)
cat. catalytic
CI chemical ionization (in MS)
coe cyclooctene
D day(s)
TLC thin-layer chromatography
DCM dichloromethane
DMA dimethylacetamide
DMF dimethylformamide
DMSO dimethyl sulfoxide
ee enantiomeric excess
EI electron impact ionization (in MS)
Ent enantiomer/enantiomerically pure
eq equivalent(s)
ESI electrospray ionization (in MS)
EtOAc ethyl acetate
GC-MS gas chromatography-coupled mass spectrometry
% by weight percent by weight
h hour(s)
HPLC high-pressure, high-performance liquid chromatography
ID internal diameter
iPrOAc isopropyl acetate
iPrOH isopropanol
conc. concentrated
l litres
LC-MS liquid chromatography-coupled mass spectrometry
LDA lithium diisopropylamide
LiHMDS lithium bis(trimethylsilyl)amide
min minute(s)
MS mass spectrometry
MTBE 2-methoxy-2-methylpropane
NMR nuclear magnetic resonance spectrometry
NMP N-methyl-2-pyrrolidone
org. organic
Ph phenyl
pTsOH p-toluenesulfonic acid
R_{f} retention index (in TLC)
RP-HPLC reversed phase high performance liquid chromatography
RRT relative retention time
Rₜ retention time
RT room temperature
TESCl chlorotriethylsilane
THF tetrahydrofuran
v/v volume-to-volume ratio (of a solution)
Tᵢₙₜ internal temperature
Tₒᵤₜ outside temperature
DM water demineralized water
aq. aqueous, aqueous solution

Analytical Methods:

Method A

High-performance liquid chromatograph with thermostated column oven, UV detector and data evaluation system, measurement wavelength 228 nm, range: 6 nm, oven temperature 25° C., column: Chiralpak AD-H, length: 250 mm, internal diameter: 4.6 mm, particle size: 5 μm, mobile phase: A: n-heptane, B: iso-propanol+0.1% diethylamine, gradient programme: start 1 ml/min 80% eluent A, 20% eluent B; 16 min 1 ml/min 40% eluent A, 60% eluent B. Sample solvent: ethanol+0.1% diethylamine, analysis solution: about 1.0 mg/ml of the substance, dissolve with sample solvent, injection volume: 10 μl

Rₜ: enantiomer 1: 7.6 min, enantiomer 2: 8.5 min

Method B

High-performance liquid chromatograph with thermostated column oven, UV detector and data evaluation system, measurement wavelength 206 nm, range: 6 nm, oven temperature 30° C., column: Chiralpak AD-H, length: 250 mm, internal diameter: 4.6 mm, particle size: 5 μm, mobile phase: A: n-heptane, B: ethanol +0.1% diethylamine, gradient programme: start 1 ml/min 70% eluent A, 30% eluent B; 12 min 1 ml/min 40% eluent A, 60% eluent B. Sample solvent: ethanol+0.1% diethylamine, analysis solution: about 1.0 mg/ml of the substance, dissolve with sample solvent, injection volume: 5 μl Rₜ: enantiomer 1: 5.8 min (RRT 1.00), enantiomer 2: 7.2 min RRT 1.25

Method C

High-performance liquid chromatograph with thermostated column oven, UV detector and data evaluation system, measurement wavelength 204 nm, range: 6 nm, oven temperature 45° C., column: Chiralpak AD-H, length: 250 mm, internal diameter: 4.6 mm, particle size: 5 μm, mobile phase: A: n-heptane, B: ethanol +0.2% trifluoroacetic acid+0.1% diethylamine, gradient programme: 1.5 min 1 ml/min 60% eluent A, 40% eluent B; sample solvent: ethanol, analysis solution: about 1.0 mg/ml of the substance, dissolve with sample solvent, injection volume: 10 μl

Rₜ: enantiomer 1 2.9 min RRT 1.00 enantiomer 2 3.7 min RRT 1.28

Method D

High-performance liquid chromatograph with thermostated column oven, UV detector and data evaluation system, measurement wavelength 230 nm, range: 6 nm, oven temperature 40° C., column: Chiralpak AD-H, length: 250 mm, internal diameter: 4.6 mm, particle size: 5 μm, mobile phase: A: n-heptane, B: ethanol +0.1% diethylamine, gradient programme: 1 min 1 ml/min 70% eluent A, 30% eluent B; sample solvent: ethanol+0.1% diethylamine, analysis solution: about 2.0 mg/ml of the substance, dissolve with sample solvent, injection volume: 10 μl

Rₜ: enantiomer 1: 4.9 min (RRT 1.00), enantiomer 2: 5.7 min (RRT 1.16)

Method E

High-performance liquid chromatograph with thermostated column oven, UV detector and data evaluation system, measurement wavelength 226 nm, range: 6 nm, oven temperature 35° C., column: Chiralpak IB, length: 250 mm, internal diameter: 4.6 mm, particle size: 5 μm, mobile phase: A: n-heptane, B: isopropanol+0.1% ethanolamine, gradient programme: 1.5 min 1 ml/min 80% eluent A, 20% eluent B; sample solvent: n-heptane:isopropanol 1:1, analysis solution: about 2.0 mg/ml of the substance, dissolve with sample solvent, injection volume: 10 μl

Rₜ: enantiomer 1: 4.1 min, enantiomer 2: 4.5 min

Method F

Variant 1:

High-performance liquid chromatograph with thermostated column oven, UV detector and data evaluation system, measurement wavelength 228 nm, range: 6 nm, oven temperature 40° C., column: Chiralpak OJ-H, length: 250 mm, internal diameter: 4.6 mm, particle size: 5 μm, mobile phase: A: n-heptane, B: ethanol +0.1% diethylamine, gradient programme: 1 min 1 ml/min 60% eluent A, 40% eluent B; sample solvent: ethanol, analysis solution: about 1.5 mg/ml of the substance, dissolve with sample solvent, injection volume: 10 μl

Rₜ: enantiomer 1: 10.68 min, enantiomer 2: 12.13 min

Variant 2:

High-performance liquid chromatograph with thermostated column oven, UV detector and data evaluation system, measurement wavelength 228 nm, range: 6 nm, oven temperature 40° C., column: Lux 3μ Cellulose-3 (Phenomenex), length: 150 mm, internal diameter: 4.6 mm, particle size: 3 μm, mobile phase: A: n-heptane, B: ethanol+0.1% diethylamine, gradient programme: 1 min 1 ml/min 75% eluent A, 25% eluent B; sample solvent: ethanol, analysis solution: about 1.0 mg/ml of the substance, dissolve with sample solvent, injection volume: 10 μl

Rₜ: enantiomer 1: 7.6 min, enantiomer 2: 8.6 min

Method G

High-performance liquid chromatograph with thermostated column oven, UV detector and data evaluation system, measurement wavelength 226 nm, range: 6 nm, oven temperature 30° C., column: Chiralpak AD-H, length: 250 mm, internal diameter: 4.6 mm, particle size: 5 μm, mobile phase: A: n-heptane, B: isopropanol+0.1% diethylamine, gradient programme: 1 min 1 ml/min 96% eluent A, 4% eluent B; sample solvent: isopropanol+0.1% diethylamine, analysis solution: about 2.0 mg/ml of the substance, dissolve with sample solvent, injection volume: 10 μl

Rₜ: enantiomer 1: 8.6 min, enantiomer 2: 10.7 min

Method H

Instrument: Waters ACQUITY SQD UPLC System; column: Waters Acquity UPLC HSS T3 1.8 μm 50×1 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; oven: 50° C.; flow rate: 0.40 ml/min; UV detection: 210 nm.

Method I

Instrument: Waters ACQUITY SQD UPLC System; column: Waters Acquity UPLC HSS T3 1.8 μm 50×1 mm; eluent A: 1 l water+0.25 ml 99% formic acid, eluent B: 1 l acetonitrile+0.25 ml 99% formic acid; gradient: 0.0 min 95% A→6.0 min 5% A→7.5 min 5% A; oven: 50° C.; flow rate: 0.35 ml/min; UV detection: 210 nm.

Method J

Instrument: Micromass Quattro Premier with Waters UPLC Acquity; column: Thermo Hypersil GOLD 1.9μ 50×1 mm; eluent A: 1 l water+0.5 ml 50% formic acid, eluent B: 1 l acetonitrile+0.5 ml 50% formic acid; gradient: 0.0 min 97% A→0.5 min 97% A→3.2 min 5% A→4.0 min 5% A; oven: 50° C.; flow rate: 0.3 ml/min; UV detection: 210 nm.

Method K

MS instrument: Waters (Micromass) QM; HPLC instrument: Agilent 1100 Series; column: Agilent ZORBAX Extend-C18 3.0×50 mm 3.5-micron; eluent A: 1 l water+0.01 mol ammonium carbonate, eluent B: 1 l acetonitrile; gradient: 0.0 min 98% A→0.2 min 98% A→3.0 min 5% A→4.5 min 5% A; oven: 40° C.; flow rate: 1.75 ml/min; UV detection: 210 nm

Method L

MS instrument type: Waters Synapt G2S; UPLC instrument type: Waters Acquity I-CLASS; column: Waters, HSST3, 2.1×50 mm, C18 1.8 μm; eluent A: 1 l water+0.01% formic acid; eluent B: 1 l acetonitrile+0.01% formic acid; gradient: 0.0 min 2% B→2.0 min 2% B→13.0 min 90% B→15.0 min 90% B; oven: 50° C.; flow rate: 1.20 ml/min; UV detection: 210 nm.

Method M

High-performance liquid chromatograph with thermostated column oven, UV detector and data evaluation system, measurement wavelength 226 nm, range: 40 nm. Column: Zorbax Bonus-RP, length: 150 mm, internal diameter: 3.0 mm, particle size: 3.5 μm, mobile phase: A: water+0.1% TFA, B: ACN+0.1% TFA/methanol=2+1, gradient programme: 0.0 min 50% B→12.0 min 70% B→17.0 min 90% B→25.0 min 90% B; flow rate: 0.60 ml/min; sample solvent: isopropanol+0.1% diethylamine, analysis solution: dissolve about 35 mg of the substance in 25 ml of ACN and make up to 50 ml with water+0.1% TFA (0.7 mg/ml); injection volume: 3 μl

Method N

High-performance liquid chromatograph with thermostated column oven, UV detector and data evaluation system, measurement wavelength 210 nm. Column: XBridge BEH Phenyl length: 50 mm, internal diameter: 4.6 mm, particle size: 2.5 μm, mobile phase: A: 0.66 g of (NH₄)₂HPO₄ and 0.58 g of (NH₄)H₂PO₄ in 1 l of millipore water; B: ACN, gradient programme: 0.0 min 95% B→8.3 min 80% B→11.0 min 80%; flow rate: 1.2 ml/min; sample solvent: ACN+water, injection volume: 3 μl.

Starting Materials and Intermediates

### Example 1

### 2-(4-Cyanophenyl)ethyl 4-methylbenzenesulfonate

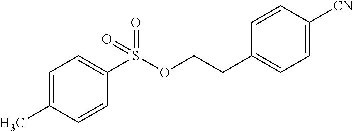 [SMILES: Cc1ccc(S(=O)(=O)OCCc2ccc(C#N)cc2)cc1]

In a 40 l reaction vessel, 12.6 l of tetrahydrofuran and 0.62 kg (11.05 mol) of potassium hydroxide (powder, 85%) were cooled to −10° C., and a solution of 813.3 g (5.53 mol) of 4-(2-hydroxyethyl)benzonitrile in 1.2 l of tetrahydrofuran was added within 13 min. Subsequently, 1.370 kg (7.18 mol) of 4-toluenesulfonyl chloride was added in several portions; the mixture was stirred at −10° C. for 20 min, heated to 22° C. and stirred at 22° C. for 1.5 h. 12.21 of water and 12.21 of dichloromethane were added, the mixture was stirred for 20 min, and the organic phase was separated off. The aqueous phase was washed with 12.2 l of dichloromethane, and the combined organic phases were washed with 12.2 l of saturated aqueous ammonium chloride solution.

The organic phases in two batches were concentrated under reduced pressure at 45° C. to 8.75 l, and the residue was metered into 40.71 of cyclohexane within 10 min. The vessel was rinsed with 11 of dichloromethane, and the rinse liquid was added to the cyclohexane. The mixture was concentrated to 24.41 at 41° C. under reduced pressure; 24.41 of cyclohexane were added and the mixture was concentrated again to 24.4 l at 41° C. under reduced pressure. The suspension was cooled to 22° C. and stirred for 30 min, and the solids were filtered off, washed with 8.2 l of cyclohexane and dried at 40° C. in a vacuum drying cabinet.

Yield: 2.82 kg; 84.6% of theory.

¹H-NMR, DMSO: 2.41 (s, 3H), 2.99 (t, 2H), 4.29 (t, 2H), 7.21-7.42 (dd, 4H), 7.52-7.72 (dd, 4H) LC-MS (Method H): Rₜ=1.03 min, 302.1 [M+H]⁺

### Example 2

### 4-(2-{[2-(2-Methoxyphenyl)ethyl]amino}ethyl)benzonitrile

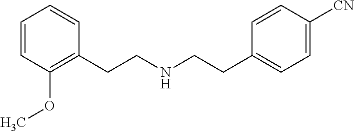 [SMILES: COc1ccccc1CCNCCc1ccc(C#N)cc1]

In a reaction vessel, 1.507 kg (5.00 mol) of cyanophenethyl tosylate (Example 1) were suspended in 3.8 l of tetrahydrofuran, and heated under reflux (about 77° C.) together with 2.27 kg (15.0 mol) of 2-methoxyphenethylamine and 1.012 kg (10.0 mol) of triethylamine for 2 h. The mixture was cooled to 50° C., and 10.71 of water were added. The solvent was distilled off under reduced pressure until only water remained. The residue was cooled to 22° C., and 6.78 l of hydrochloric acid (25%) were added within 40 min. The mixture was stirred for 30 min, and the solids were filtered off with suction and washed with 1 l of water.

The solids in two batches were stirred with 15 l of water for 30 min, the solids were filtered off with suction and washed with 7.5 l of water, and the procedure was repeated. The moist product was stirred with 7.5 l of ethyl acetate at 50° C. for 1.5 h, cooled to 22° C., stirred at 22° C. for 1 h, filtered off, washed with 5 l of ethyl acetate and dried at 40° C. in a vacuum drying cabinet to give 2.13 kg. The dry product was stirred in 2.2 l of ethyl acetate and 5.38 l of hydrochloric acid (15%), filtered off with suction, washed with 2.15 l of water and dried at 40° C. in a vacuum drying cabinet to give 1.63 kg of hydrochloride.

The hydrochloride was dissolved in 8.25 l of dichloromethane and 8.25 l of water, 45% sodium hydroxide solution was used to adjust the pH to from 13 to 14, the phases were separated and the organic phase was washed with 2.75 l of water. The organic phase was concentrated at 40° C. under reduced pressure, 3 l of dichloromethane were added, and the mixture was concentrated again to give 1.44 kg of oil.

Yield: 1.44 kg; 51.5% of theory.

¹H-NMR, DMSO: 2.59-2.72 (m, 4H), 2.77 (s, 4H), 3.77 (s, 3H), 6.80-6.98 (m, 2H), 7.08-7.21 (m, 2H), 7.41 (d, 2H), 7.72 (d, 2H)

LC-MS (Method H): Rₜ=0.63 min, 281.2 [M+H]⁺

### Example 3

### (5R)-5-Hydroxy-5,6,7,8-tetrahydroquinoline-2-carbonitrile

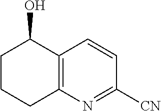 [SMILES: N#Cc1ccc2c(n1)CCC[C@H]2O]

A 30 l stainless steel reactor was initially charged with 1.0 kg of 5-oxo-5,6,7,8-tetrahydroquinoline-2-carbonitrile and 10.0 l of ethyl acetate. 1.175 kg of triethylamine were metered in at 20° C. within 15 min. 18.5 g of ruthenium-p-cymene-R,R-TsDPEN (CAS number: 192139-92-7) were added to the solution at 20° C. 1.337 kg of formic acid were metered into the solution at 0° C. to 5° C. within 1 h (evolution of gas). The reaction was stirred at internal temperature 40° C. for 4 h. The monitoring of the reaction showed complete conversion after only 2 h at 40° C. (laboratory HPLC). The reaction mixture was cooled down to 20° C. and stirred at 20° C. overnight for release of gas. For workup, the reaction mixture was admixed with 4.11 of ethyl acetate and 4.11 of 1 N hydrochloric acid, and stirred for a further 15 min. The phases were separated. About 13.9 l of a dark brown organic upper phase were obtained. The product-containing upper phase was admixed with 13.9 l of n-heptane. The mixture was concentrated under reduced pressure (about 800 mbar, outside temperature about 40° C.) within about 2.5 h, until an amount of about 17.6 l of distillate was attained. Another 13.9 l of n-heptane were added, and the mixture was concentrated again within about 2.5 h until an amount of about 17.6 l of distillate had been attained (final volume of the mixture about 7 l). The mixture was cooled down to about 20° C. and stirred at 20° C. overnight. The product was isolated by filtration, and the crystals were washed twice with 3.7 l each time of n-heptane. The moist product was dried to constant mass in a vacuum drying cabinet at outside temperature about 40° C. for about 17 h.

Yield: 0.975 kg; 96% of theory.

¹H-NMR, DMSO (NBR 305-22-1): 1.60-1.85 (m, 2H), 1.90-2.05 (m, 2H), 2.75-2.93 (m, 2H), 4.65-4.70 (m, 1H), 5.62 (d, 1H), 7.85 (d, 1H), 8.0 (d, 1H)

LC-MS (Method H): Rₜ=0.52 min 175.1 [M+H]⁺

Enantiomeric purity (HPLC Method D): 98.03% ee

### Example 4

### (5S)-5-{[2-(4-Cyanophenyl)ethyl][2-(2-methoxyphenyl)ethyl]amino}-5,6,7,8-tetrahydroquinoline-2-carbonitrile

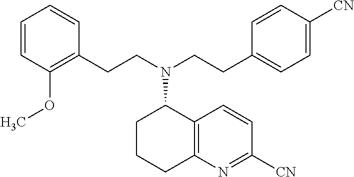 [SMILES: COc1ccccc1CCN(CCc1ccc(C#N)cc1)[C@H]1CCCc2nc(C#N)ccc21]

In a 6 l flask, a solution of 121.2 g (0.696 mol) of (5R)-5-hydroxy-5,6,7,8-tetrahydroquinoline-2-carbonitrile (Example 3) in 220 ml of dichloromethane and 211.2 g (2.09 mol) of diisopropylamine under argon was cooled to −76° C. Within 85 min, 333.6 g (1.18 mol) of trifluoromethanesulfonic anhydride was metered in at −76° C. to −69° C. and rinsed in with 20 ml of dichloromethane, and the mixture was stirred for 20 min. Subsequently, within 35 min, a solution of 292.5 g (1.044 mol) of 4-(2-{[2-(2-methoxyphenyl)ethyl]amino}ethyl)benzonitrile (Example 3) in 720 ml of dichloromethane was metered in at −75° C. to −67° C. and rinsed in with 80 ml of dichloromethane, and the mixture was stirred for 2 h. 172.1 g (1.19 mol) of oxalic acid was added to the reaction mixture, the cooling bath was removed and the mixture was stirred overnight. 216 g of kieselguhr were added, the reaction mixture was adjusted to a temperature of 0° C. to 5° C. and stirred for 30 min, and the solids were filtered off with suction. The filtercake was washed with 1680 ml of cold dichloromethane, and the filtrate was washed with 2 l of water. The organic phase was admixed with 2 l of water and adjusted to pH=8 with 40 ml of aqueous ammonia solution (27%), and the aqueous phase was separated off. The organic phase was concentrated on a rotary evaporator at 40° C. under reduced pressure to give an oil (380.3 g). The oil was dissolved in 758 ml of ethanol under reflux, cooled to 40° C., seeded with product and cooled further to room temperature. The solids were filtered off with suction, washed with 300 ml of ethanol and dried in a vacuum drying cabinet at 25° C. in a stream of nitrogen.

Yield: 167.8 g; (55.2% of theory)

Enantiomeric purity (HPLC Method F): 87.9% ee

LC-MS (Method H): Rₜ=1.31 min 437.2 [M+H]⁺

### Example 5

### 4-(2-{[2-(2-Hydroxyphenyl)ethyl]amino}ethyl)benzonitrile

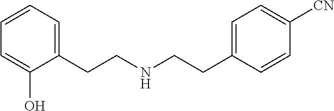 [SMILES: N#Cc1ccc(CCNCCc2ccccc2O)cc1]

In a 2 l flask, 155.6 g (1.167 mol) of aluminium chloride and 429.5 g (2.122 mol) of dodecyl mercaptan were stirred until dissolution (15 min). Within 30 min, a solution of 119.0 g (0.424 mol) of 4-(2-{[2-(2-methoxyphenyl)ethyl]amino}ethyl)benzonitrile (Example 2) in 418 ml of toluene was metered in at 10°-20° C. It was rinsed in with 42 ml of toluene, and the mixture was stirred at 40° C. overnight. The resultant solids were filtered off with suction, washed with 530 ml of dichloromethane, stirred with 800 ml of dichloromethane and filtered off with suction. The moist product was dissolved in 835 ml of tetrahydrofuran, and 526 ml (2.33 mol) of saturated (360 g/1) sodium potassium tartrate solution were added while cooling. The biphasic mixture was filtered with suction, and the solids were stirred with 1 l of ethyl acetate and filtered off with suction. The purified solids were suspended in 835 ml of tetrahydrofuran and stirred with 526 ml (2.33 mol) of saturated (360 g/1) sodium potassium tartrate solution for 30 min. The solids were filtered off with suction from the product-containing filtrate and washed with 200 ml of tetrahydrofuran. The product-containing filtrates were combined, and the organic phase was separated off and concentrated. The residue was dissolved in 835 ml of dichloromethane, alkalized with 31 ml of aq. ammonia solution (27%) and washed three times with 309 ml each time of water. The combined organic phases were washed with 155 ml of dichloromethane, and the combined organic phases were concentrated to give an oil.

Crude yield: 70.8 g; 62.6% of theory.

LC-MS (Method I): Rₜ=1.20 min, 267.2 [M+H]⁺

### Example 6

### 4-(2-{[2-(2-{[tert-Butyl(dimethyl)silyl]oxy}phenyl)ethyl]amino}ethyl)benzonitrile

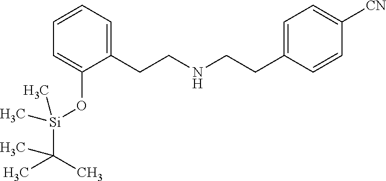 [SMILES: CC(C)(C)[Si](C)(C)Oc1ccccc1CCNCCc1ccc(C#N)cc1]

Method A:

In a 2 l flask, 68.99 g (0.26 mol) of 4-(2-{[2-(2-hydroxyphenyl)ethyl]amino}ethyl)benzonitrile (Example 5) were dissolved in 690 ml of dichloromethane. While cooling, 68.43 g (0.44 mol) of tert-butyldimethylsilyl chloride and 26.5 g (0.39 mol) of imidazole were added at 23° C. to 33° C., and the mixture was stirred at room temperature for 16 h. Subsequently, a solution of 60.85 g of potassium carbonate in 420 ml of water was added, the organic phase was washed three times with 350 ml each time of water, the combined organic phases were washed with 80 ml of dichloromethane, and the combined organic phases were dried with sodium sulfate and concentrated on a rotary evaporator at 35° C. to give 116.5 g of crude product.

Crude yield: 116.5 g; 118% of theory.

LC-MS (Method J): Rₜ=2.21 min, 381.3 [M+H]⁺, 382.2

Method B:

To a solution of cyanophenethyl tosylate (10 g, 33.2 mmol, 1.0 eq.) in THF (130 ml) were added, at a temperature of 25-35° C., 2-(2-aminoethyl)phenol (9.1 g, 66.4 mmol, 2.0 eq.) and triethylamine (13.8 ml, 99.5 mmol, 3.0 eq.). Subsequently, the reaction mixture was heated to reflux for 46 h. Subsequently, the THF was removed under reduced pressure at a temperature of less than 60° C., and the remaining amount of crude material was admixed with DCM (50 ml). The solution was then washed with saturated sodium bicarbonate solution (2×50 ml), and the organic phase was concentrated at a temperature of less than 45° C.

To this DCM solution (40 ml) were added imidazole (6.8 g, 99.5 mmol, 3.0 eq.) and then, in portions, tert-butyl-dimethylsilyl chloride (14.0 g, 92.9 mmol, 2.8 eq.). The reaction mixture was subsequently stirred at 25-35° C. for 2 h. After the reaction had concluded, the reaction mixture was washed with water (2×100 ml). By means of distillation under reduced pressure, the solvent was changed to methanol (100 ml), and the mixture was heated to 65° C. Subsequently, oxalic acid (4.5 g, 49.7 mmol, 1.5 eq) was added and the mixture was stirred at 50-55° C. for 1-2 h. The reaction mixture was cooled down gradually to 5-10° C. and stirred for a further 1-2 hours. The solids were then filtered off and washed with methanol (2×20 ml). Subsequently, the filter residue was suspended in DCM/water (137 ml of each) and stirred at 25-35° C. for several hours. Subsequently, 45% NaOH (4.5 ml) was added in order to attain a pH of 10.5-12.5. After 1 hour, a further 70 ml of water were added and the phases were separated. The organic phase was concentrated under reduced pressure. The resultant residue corresponds to the target substance (6.78 g, 37%).

Yield: 6.78 g; 37% of theory.

Purity (area): 91.3% (Method N, Rₜ 11 min)

### Example 7

### (5S)-5-{[2-(2-{[tert-Butyl(dimethyl)silyl]oxy}phenyl)ethyl][2-(4-cyanophenyl)ethyl]amino}-5,6,7,8-tetrahydroquinoline-2-carbonitrile

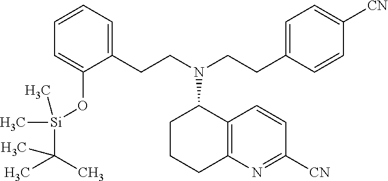 [SMILES: CC(C)(C)[Si](C)(C)Oc1ccccc1CCN(CCc1ccc(C#N)cc1)[C@H]1CCCc2nc(C#N)ccc21]

In a 11 flask, a solution of 15.0 g (86.1 mmol) of (5R)-5-hydroxy-5,6,7,8-tetrahydroquinoline-2-carbonitrile (Example 3) in 250 ml of dichloromethane and 36.2 ml (0.26 mol) of diisopropylamine under argon was cooled to −76° C. Within 30 min, 24.6 ml (0.15 mol) of trifluoromethanesulfonic anhydride were metered in at −74° C. to −68° C., and the mixture was stirred for 30 min. Subsequently, within 46 min, a solution of 49.2 g (0.13 mol) of 4-(2-{[2-(2-{[tert-butyl(dimethyl)silyl]oxy}phenyl)ethyl]amino}ethyl)benzonitrile (Example 6) in 100 ml of dichloromethane was metered in at −75° C. to −72° C. and rinsed in with 20 ml of dichloromethane, and the mixture was stirred for 2 h. 9.93 g (86.1 mmol) of 85% phosphoric acid were added to the reaction mixture, and the reaction mixture was washed twice at room temperature with 500 ml each time of water. The combined aqueous phases were washed with 300 ml of dichloromethane, and the combined organic phases were concentrated on a rotary evaporator at 40° C. under reduced pressure to give an oil (96.6 g).

The oil was dissolved in 50 ml of dichloromethane and filtered through 150 g of silica gel, and the product was eluted with 800 ml of ethyl acetate/n-hexane in a ratio of 1:2. The product solution was concentrated on a rotary evaporator at 40° C. under reduced pressure to give an oil (79.3 g).

The product was dissolved in 50 ml of dichloromethane and filtered through 150 g of silica gel, and was eluted with 750 ml of ethyl acetate/n-hexane in a ratio of 1:2. The eluate was concentrated on a rotary evaporator at 35° C. to give 48.2 g of crude product.

Crude yield: 48.2 g; 104% of theory.

LC-MS (Method K): Rₜ=3.70 min 537.2 [M+H]⁺

### Example 8

### (5S)-5-{[2-(4-Cyanophenyl)ethyl][2-(2-hydroxyphenyl)ethyl]amino}-5,6,7,8-tetrahydroquinoline-2-carbonitrile

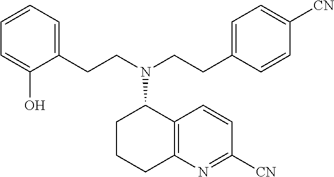 [SMILES: N#Cc1ccc(CCN(CCc2ccccc2O)[C@H]2CCCc3nc(C#N)ccc32)cc1]

In a 1 l flask, 48.2 g (not more than 86.1 mmol) of (5S)-5-{[2-(2-{[tert-butyl(dimethyl)silyl]oxy}phenyl)ethyl][2-(4-cyanophenyl)ethyl]amino}-5,6,7,8-tetrahydroquinoline-2-carbonitrile (Example 7) were suspended in 550 ml of methanol, and 101.8 g of conc. hydrochloric acid were added. The solution was stirred at RT overnight, 150.9 g of 30% ammonia solution were added while cooling, and the mixture was concentrated on a rotary evaporator at 40° C. The solid residue was stirred in 480 ml of demineralized water and 250 ml of dichloromethane at RT for 30 min; the organic lower phase was washed with 450 ml of water and concentrated on a rotary evaporator at 35° C. to give 27.9 g.

Yield: 27.9 g; 76.8% of theory.

Enantiomeric purity (HPLC Method A): 91.4% ee

The residue was heated under reflux in 100 ml of methanol/10 ml of demineralized water, and the suspension was cooled down to room temperature and stirred for 2 h. The solids were filtered off with suction, washed with 15 ml of methanol and dried in a vacuum drying cabinet at 50° C.

Yield: 12.96 g; 35.6% of theory.

Enantiomeric purity (HPLC Method A): 98.6% ee

### Example 9

### (5S)-5-{[2-(4-Carboxyphenyl)ethyl][2-(2-hydroxyphenyl)ethyl]amino}-5,6,7,8-tetrahydroquinoline-2-carboxylic acid

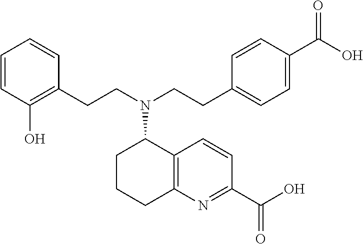 [SMILES: O=C(O)c1ccc(CCN(CCc2ccccc2O)[C@H]2CCCc3nc(C(=O)O)ccc32)cc1]

Method A:

In a 25 ml flask, 1.0 g (2.4 mmol) of (5S)-5-{[2-(4-cyanophenyl)ethyl][2-(2-hydroxyphenyl)ethyl]amino}-5,6,7,8-tetrahydroquinoline-2-carbonitrile (Example 8) was suspended in 5.79 g of conc. hydrochloric acid and stirred at 100° C. for 24 h. The reaction solution was used directly in the next reaction stage (Example 10).

Method B:

In a 6 l flask with gas scrubber (contents: 600 g of ethanolamine, 1200 g of 5% sodium hydroxide solution, 1200 g of isopropanol and about 0.5 g of bromothymol blue), 332.5 g (0.76 mol) of (5S)-5-{[2-(4-cyanophenyl)ethyl][2-(2-methoxyphenyl)ethyl]amino}-5,6,7,8-tetrahydroquinoline-2-carbonitrile (Example 4) in 3210 g (2.15 ml) of 48% hydrobromic acid were heated to 108° C. and stirred for 24 h. The solution was cooled to 25° C. and washed twice with 650 ml each time of dichloromethane. The lower aqueous product phase was used in the next stage (Example 10). A sample was purified for analytical purposes.

LC-MS (Method H): Rₜ=0.73 min, 461.2 [M+H]+

### Example 10

### Butyl (5S)-5-({2-[4-(butoxycarbonyl)phenyl]ethyl}[2-(2-hydroxyphenyl)ethyl]amino)-5,6,7,8-tetrahydroquinoline-2-carboxylate

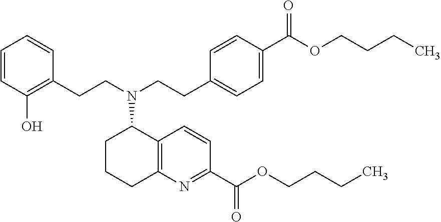 [SMILES: CCCCOC(=O)c1ccc(CCN(CCc2ccccc2O)[C@H]2CCCc3nc(C(=O)OCCCC)ccc32)cc1]

Method A:

In a 6 l flask, 1.5 l of n-butanol were added to the aqueous product phase ((5S)-5-{[2-(4-carboxyphenyl)ethyl][2-(2-hydroxyphenyl)ethyl]amino}-5,6,7,8-tetrahydroquinoline-2-carboxylic acid (Example 9), the solution was heated to boiling, and the butanol/water mixture was distilled off with continuous addition of 6 l of butanol until a top temperature of 117° C. was attained. The mixture was cooled to room temperature, the precipitated salts were filtered off with suction, and the filtercake was washed with 600 ml of butanol. The combined filtrates were concentrated on a rotary evaporator at 65° C. under reduced pressure to give 521.7 g. The residue was stirred with 2.2 l of ethyl acetate and 1.1 l of 14% aqueous ammonia solution for 30 min, and the organic phase was separated off, washed twice with 1 l each time of water and concentrated at 40° C. on a rotary evaporator under reduced pressure to give 409.6 g of oil.

The oil was dissolved in 500 ml of dichloromethane and filtered, with a further 8 l of dichloromethane and then 2 l of methanol, through a filter covered with 1 kg of silica gel. The product solution was concentrated on a rotary evaporator to give 337.8 g of oil.

Yield: 337.8 g; 77.6% of theory.

LC-MS (Method H): Rₜ=1.34 min, 573.3 [M+H]⁺

A sample was purified for analytical purposes.

¹H-NMR, (400 MHz, CDCl3): δ=0.88-1.06 (m, 6H), 1.36-1.53 (m, 4H), 1.65-1.91 (m, 6H), 2.05-2.31 (m, 2H), 2.63-3.31 (m, 10H), 4.19-4.34 (m, 2H), 4.34-4.48 (m, 3H), 6.69-6.83 (m, 1H), 6.88-6.96 (m, 2H), 7.08-7.21 (m, 3H), 7.71-7.85 (m, 1H), 7.85-7.97 (m, 2H), 8.00-8.15 (m, 1H), 10.40-10.59 (br. s, 1H)ppm.

Method B

An inertized 21 reactor was initially charged with (5R)-5-hydroxy-5,6,7,8-tetrahydroquinoline-2-carbonitrile (Example 3) (40 g, 1.0 eq.) in dichloromethane (850 ml). Diisopropylamine (3.0 eq., 69.6 g) was added, and the solution was cooled down to Tₒᵤₜ=−90° C. At Tᵢₙₜ=−77 to −67° C., a solution of trifluoromethanesulfonic anhydride (1.5 eq., 60 ml) and dichloromethane (150 ml) was metered in within about 1 h. This was followed by stirring for a further 45 min. Then, at Tᵢₙₜ=−78 to −70° C., a solution of 4-(2-{[2-(2-{[tert-butyl(dimethyl)silyl]oxy}phenyl)ethyl]amino}ethyl)benzonitrile (Example 6) (1.05 eq., 70 g) and dichloromethane (200 ml) was metered in within about 30 min. This was followed by stirring for a further 1.5 h. Then the mixture was heated to Tᵢₙₜ=20° C. within 1 h. The second 21 reactor was initially charged with 3.6% hydrochloric acid (610 ml). The reaction mixture was added and the mixture was stirred for 5 min. The phases were allowed to settle and the aqueous phase was separated off (discarded). The organic phase was concentrated to the limit of stirrability at standard pressure and Tₒᵤₜ=60° C. 25% hydrochloric acid was added, and the mixture was distilled at standard pressure and Tₒᵤₜ=85° C. until it ran dry. Thereafter, it was heated to reflux (Tᵢₙₜ=103° C., Tₒᵤₜ=125° C.) and stirred for a further 5 h. Then the mixture was cooled to Tᵢₙₜ=40° C. and stirred for a further 14 hours. Subsequently, the resultant suspension was filtered, n-butanol (800 ml) was added to the filtrate, and the mixture was concentrated until attainment of an internal temperature of Tᵢₙₜ=88° C. n-Butanol (800 ml) was added again, and the mixture was concentrated under the same conditions to Tᵢₙₜ=90° C. n-Butanol (800 ml) was added once again, and the mixture was concentrated under the same conditions to Tᵢₙₜ=102° C. n-Butanol (800 ml) was added one last time, and the mixture was concentrated under the same conditions to the limit of stirrability. The solution was cooled down to Tᵢₙₜ=22° C. Ethyl acetate (800 ml), demineralized water (400 ml) and potassium carbonate (44 g) were added, and the mixture was stirred for a further 10 min.

The phases were allowed to settle and the aqueous phase was separated off (discarded). Demineralized water (385 ml) and sodium chloride (43 kg) were added to the organic phase, and the mixture was stirred for 10 min. The phases were allowed to settle and the aqueous phase was separated off (discarded). Demineralized water (200 ml) was added to the organic phase, and the mixture was stirred for 10 min. The phases were allowed to settle and the aqueous phase was separated off (discarded). The organic phase was concentrated to the limit of stirrability under a reduced pressure of 120 mbar and Tₒᵤₜ=45-55° C. The solution was cooled down to Tᵢₙₜ=22° C. and dispensed.

Yield: 68.3 kg of solution with a content of 23.1%, 52%

Purity (area): 66.6% (Method N, Rₜ: 11 min)

### Example 11

### Butyl (5S)-5-({2-[4-(butoxycarbonyl)phenyl]ethyl}[2-(2-{[3-chloro-4′-(trifluoromethyl)[biphenyl]-4-yl]methoxy}phenyl)ethyl]amino)-5,6,7,8-tetrahydroquinoline-2-carboxylate

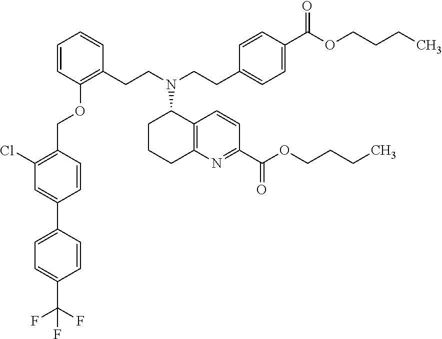 [SMILES: CCCCOC(=O)c1ccc(CCN(CCc2ccccc2OCc2ccc(-c3ccc(C(F)(F)F)cc3)cc2Cl)[C@H]2CCCc3nc(C(=O)OCCCC)ccc32)cc1]

To a solution of 337 g (0.56 mol) of butyl (5S)-5-({2-[4-(butoxycarbonyl)phenyl]ethyl}[2-(2-hydroxyphenyl)ethyl]amino)-5,6,7,8-tetrahydroquinoline-2-carboxylate (Example 10) in 3765 g of acetonitrile in a 6 l flask at room temperature was added 197.2 g (0.56 mol) of 4-(bromomethyl)-3-chloro-4′-(trifluoromethyl)[biphenyl]. 551.3 g (1.70 mol) of caesium carbonate was added to the solution and the mixture was stirred for 21 h until conversion was complete. Subsequently, the salts were filtered off with suction and washed with 600 ml of acetonitrile, and the combined filtrates were concentrated on a rotary evaporator at 40° C. to give 484.4 g of oil.

Crude yield: 484.4 g; 103% of theory.

Enantiomeric purity (HPLC Method B): 100.0% ee

LC-MS (Method L): Rₜ=14.63 min 841.36 [M+H]⁺

1. A process for preparing the compound of the formula (XII) 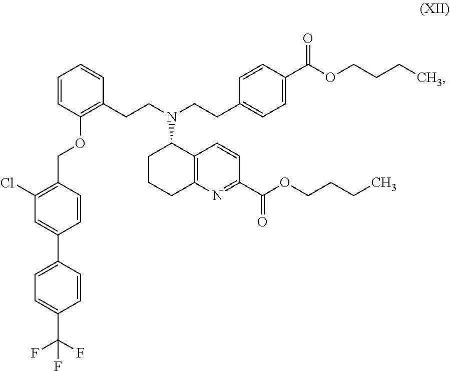 [SMILES: CCCCOC(=O)c1ccc(CCN(CCc2ccccc2OCc2ccc(-c3ccc(C(F)(F)F)cc3)cc2Cl)[C@H]2CCCc3nc(C(=O)OCCCC)ccc32)cc1]
comprising reacting the compound of the formula (X) 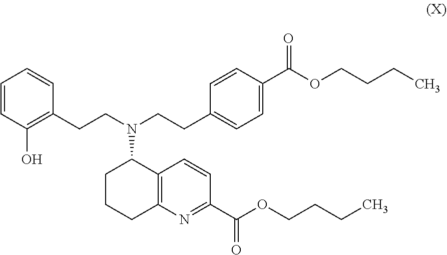 [SMILES: CCCCOC(=O)c1ccc(CCN(CCc2ccccc2O)[C@H]2CCCc3nc(C(=O)OCCCC)ccc32)cc1]
in the presence of an alkali metal carbonate, alkali metal hydroxide or tetraalkylammonium carbonate with the compound of the formula (XI) 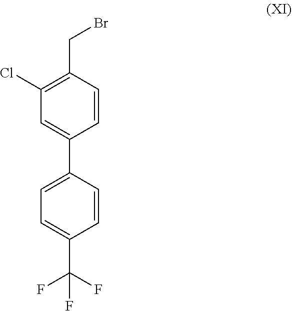 [SMILES: FC(F)(F)c1ccc(-c2ccc(CBr)c(Cl)c2)cc1]
to prepare the compound of the formula (XII).

2. The process for preparing the compound of the formula (XII) according to claim 1, wherein the alkali metal carbonate is caesium carbonate.

3. Butyl (5S)-5-({2-[4-(butoxycarbonyl)phenyl]ethyl}[2-(2-{[3-chloro-4′-(trifluoromethyl)[biphenyl]-4-yl]methoxy}phenyl)ethyl]amino)-5,6,7,8-tetrahydroquinoline-2-carboxylate of the formula (XII) 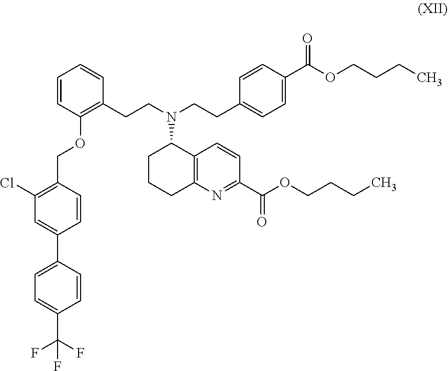 [SMILES: CCCCOC(=O)c1ccc(CCN(CCc2ccccc2OCc2ccc(-c3ccc(C(F)(F)F)cc3)cc2Cl)[C@H]2CCCc3nc(C(=O)OCCCC)ccc32)cc1]
and the salts, solvates and solvates of the salts thereof.

4. A process for preparing the compound of the formula (X), comprising reacting the compound of the formula (IX) 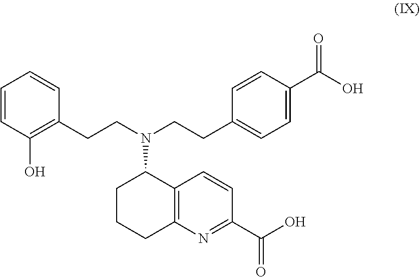 [SMILES: O=C(O)c1ccc(CCN(CCc2ccccc2O)[C@H]2CCCc3nc(C(=O)O)ccc32)cc1]
with butanol in the presence of a mineral acid.

5. A process for preparing the compound of the formula (X) 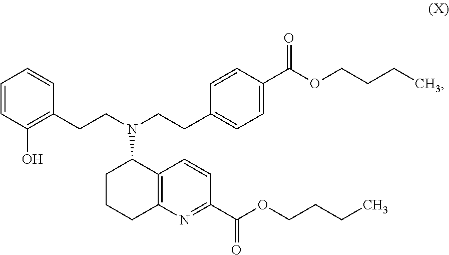 [SMILES: CCCCOC(=O)c1ccc(CCN(CCc2ccccc2O)[C@H]2CCCc3nc(C(=O)OCCCC)ccc32)cc1]
characterized in that, in a first step at a temperature of −90° C. to −50° C., it is added to the compound of the formula (III) 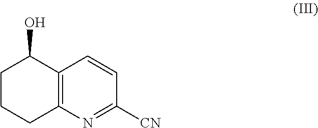 [SMILES: N#Cc1ccc2c(n1)CCC[C@H]2O]
in the presence of a base selected from a list consisting of sterically hindered secondary amines and 2,6-disubstituted pyridines, trifluoromethanesulfonic anhydride, and in a second step is reacted at a temperature of −90° C. to −50° C. with the compound of the formula (XIV-1) 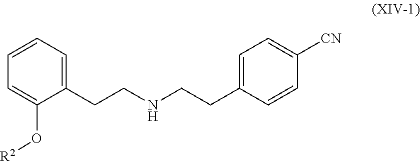
where
R² is a silyl protecting group,
in a third step the reaction product is reacted with hydrochloric acid, and in a fourth step the reaction product is reacted with butanol in the presence of a mineral acid.

6. Butyl (5S)-5-({2-[4-(butoxycarbonyl)phenyl]ethyl}[2-(2-hydroxyphenyl)ethyl]amino)-5,6,7,8-tetrahydroquinoline-2-carboxylate of the formula (X) 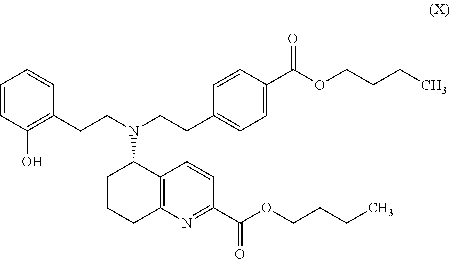 [SMILES: CCCCOC(=O)c1ccc(CCN(CCc2ccccc2O)[C@H]2CCCc3nc(C(=O)OCCCC)ccc32)cc1]
and the salts, solvates and solvates of the salts thereof.

7. A process for preparing the compound of the formula (IX), comprising reacting the compound of the formula (XVI) 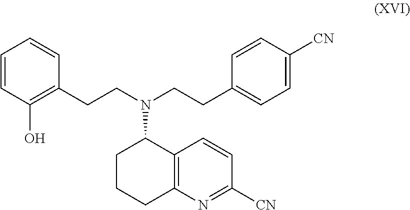 [SMILES: N#Cc1ccc(CCN(CCc2ccccc2O)[C@H]2CCCc3nc(C#N)ccc32)cc1]
with a mineral acid.

8. (5S)-5-{[2-(4-Carboxyphenyl)ethyl][2-(2-hydroxyphenyl)ethyl]amino}-5,6,7,8-tetrahydroquinoline-2-carboxylic acid of the formula (IX) 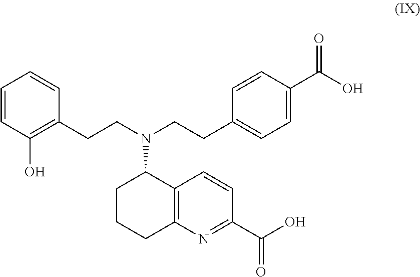 [SMILES: O=C(O)c1ccc(CCN(CCc2ccccc2O)[C@H]2CCCc3nc(C(=O)O)ccc32)cc1]
and the salts, solvates and solvates of the salts thereof.

9. A process for preparing the compound of the formula (XVI), characterized in that the compound of the formula (XV-1) 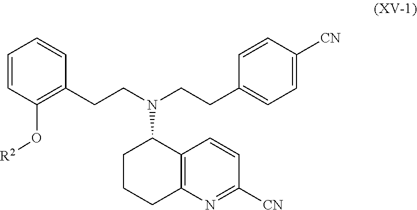
where
R² is a silyl protecting group
is reacted with a mineral acid.

10. (5S)-5-{[2-(4-Cyanophenyl)ethyl][2-(2-hydroxyphenyl)ethyl]amino}-5,6,7,8-tetrahydroquinoline-2-carbonitrile of the formula (XVI) 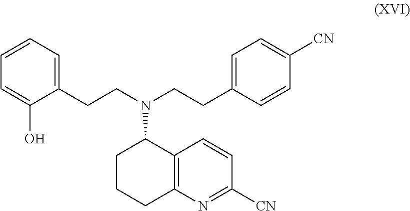 [SMILES: N#Cc1ccc(CCN(CCc2ccccc2O)[C@H]2CCCc3nc(C#N)ccc32)cc1]
and the salts, solvates and solvates of the salts thereof.

11. A process for preparing the compound of the formula (XV-1), characterized in that, at a temperature of −90° C. to −50° C. in a first step, it is added to the compound of the formula (III) 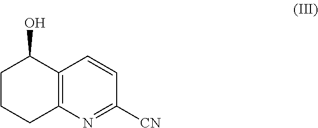 [SMILES: N#Cc1ccc2c(n1)CCC[C@H]2O]
in the presence of a base selected from a list consisting of sterically hindered secondary amines and 2,6-disubstituted pyridines and trifluoromethanesulfonic anhydride, and in a second step is reacted with the compound of the formula (XVI-1) 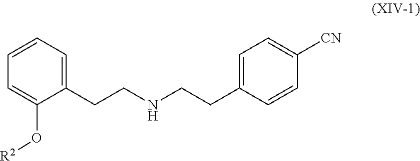
where
R² is a silyl protecting group.

12. A process for preparing the compound of the formula (XIV-1), characterized in that the compounds of the formulae (XVII) and (V) 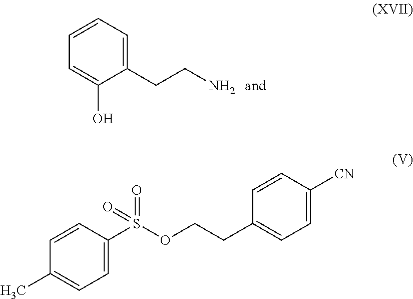 [SMILES: NCCc1ccccc1O ; Cc1ccc(S(=O)(=O)OCCc2ccc(C#N)cc2)cc1]
in a first step are coupled in the presence of an amine base, and the reaction product in a second step is reacted with the appropriate silyl chloride, likewise in the presence of an amine base.

13. A process for preparing the compound of the formula (III), characterized in that the compound of the formula (II) 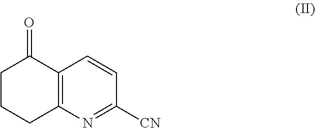 [SMILES: N#Cc1ccc2c(n1)CCCC2=O]
is reacted with a tertiary amine base, ruthenium-p-cymene-R,R-TsDPEN and formic acid to give the compound of the formula (III)

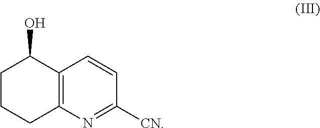 [SMILES: N#Cc1ccc2c(n1)CCC[C@H]2O]

14. (5R)-5-Hydroxy-5,6,7,8-tetrahydroquinoline-2-carbonitrile of the formula (III) 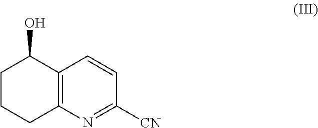 [SMILES: N#Cc1ccc2c(n1)CCC[C@H]2O]
and the salts, solvates and solvates of the salts thereof.

15. A process for preparing the compound of the formula (V), comprising reacting the compound of the formula (IV) 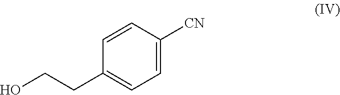 [SMILES: N#Cc1ccc(CCO)cc1]
with potassium hydroxide and 4-toluenesulfonyl chloride in an inert solvent.
